(19) United States
(12) Patent Application Publication
(10) Pub. No.: US 2026/0115101 A1
(43) Pub. Date: Apr. 30, 2026

(54) METHOD, DEVICE, AND SYSTEM FOR TRACKING MEDICATIONS

(71) Applicant: Home Service Collaboration, LLC, Blue Ash, OH (US)

(72) Inventors: Stephen Rosedale, Cincinatti, OH (US); Ronald Wilheim, Cincinatti, OH (US)

(73) Assignee: Home Service Collaboration, LLC, Blue Ash, OH (US)

(21) Appl. No.: 19/361,064

(22) Filed: Oct. 17, 2025

Related U.S. Application Data
(60) Provisional application No. 63/713,720, filed on Oct. 30, 2024.

(51) Int. Cl. A61J 7/00; A61G 12/00; G06K 7/14; G16H 20/13

(52) U.S. Cl. CPC A61J 7/0076; A61G 12/001; G16H 20/13; A61G 2203/20; A61J 2205/10; G06K 7/1413

(57) ABSTRACT

A method of tracking medications to be administered includes assigning, in a computing device, a medication to a slot in a cart insert for a medication cart. Medication tracking data is recorded in a database for the location of that medication and the identity of an individual to whom the medication is to be administered. The cart insert with the medications packed therein is loaded into a medication cart. A computing device retrieves the medication tracking data from the database, and upon selection in a computing device of the individual and medication to be administered, the computing device identifies the location of the medication in the cart by illuminating lights on the front of the drawer of the cart at which the medication is located, and lights adjacent a row and a column of slots at the intersection of which is found the slot containing the selected medication.

### CROSS-REFERENCE TO RELATED APPLICATIONS

The present application claims the benefit of priority under 35 U.S.C. § 119 from U.S. Provisional Patent Application Ser. No. 63/713,720 entitled “Method, Device, and System for Tracking Medication,” filed on Oct. 30, 2024, the disclosure of which is hereby incorporated by reference in its entirety for all purposes.

### FIELD

This disclosure generally relates to a method, device, and system for tracking and dispensing medications.

### BACKGROUND

Medication dispensing in facilities, such as nursing homes, hospitals, and other healthcare facilities can be a complex and time consuming process. In such institutional settings with numerous residents or patients, prescriptions for medications to be administered to the residents or patients are often filled by a central or local pharmacy at the same time, aggregated into a single bag, box or tote, shipped to the health care facility all mixed together, and left for nurses to sort out at the health care facility when the medication arrives thereto. Nurses and other qualified health care providers need to find each resident's or patient's medication mixed among the others, ensure the correct medications are given to the correct resident or patient, and often have to provide medications to multiple residents or patients in successive order from a single medication cart. Such efforts routinely involve nurses needing to look through a tray on a medical cart that contains multiple medications for numerous residents or patients, in order to find the correct medications prescribed to a given resident or patient and dispensing them accordingly, all of which can be time consuming and inefficient as currently practiced. Accordingly, there is a need for an improved method and system of tracking and dispensing medications to residents of nursing homes or patients in a hospital that is quicker, more accurate, and more efficient.

### SUMMARY

According to certain aspects of the present disclosure a method of tracking medications to be administered is provided. The method includes assigning, in a pharmacy computing device, a medication of a resident to a desired slot in a cart insert having a plurality of slots defined therein arranged into a plurality of rows and columns. The method includes recording, in a database of a medication tracking service in communication with the pharmacy computing device, medication tracking data including a location of the assigned slot to which the medication was assigned in the cart insert, and an identification of the resident to whom the assigned medication belongs. The method includes packing the first medication into the assigned slot of the cart insert. The method includes allocating, in a facility computing device, the cart insert to a desired drawer position within a desired drawer of a medication cart. The method includes recording, in the database in communication with the facility computing device, additional medication tracking data, including identification of each of the specific drawer of the cart and the drawer position within the cart to which the cart insert is allocated. The method includes loading the cart insert into the drawer of the cart at the allocated drawer position. The method includes signaling the location at which the selected medication to be administered to the resident is located within the medical cart by: transmitting the medication tracking data from the database to the facility computing device, determining, by the facility computing device based on the medication tracking data, each of the drawer and drawer position in the cart in which the cart insert containing the mediation is loaded, the slot position within the cart insert into which the medication is packed, and a relative location of the slot in relation to a plurality of lights disposed along each of a front and side edge of the drawer in which the slot is located, and illuminating, by the facility computing device, a group of lights from each of the plurality of lights disposed along the font edge of the drawer that are visible when the drawer is in a closed position and a plurality of lights disposed along the side edge of the drawer, which illuminated group of lights are positioned at locations on the respective side and front edges of the drawer adjacent the respective row and column at which the slot is located in the cart insert within the drawer of the cart, at the intersection of which row and column the slot containing the medication is located.

According to certain aspects of the present disclosure, a smart medication cart for use in administering medication to residents of a facility is provided. The smart medication cart includes a cabinet defining a countertop. The smart medication cart includes a plurality of wheels affixed to a bottom of said cabinet. The smart medication cart includes a plurality of slidable drawers disposed within said cabinet and configured to be slidably moved between an opened position and a closed position, and to hold one or more selectively removable cart inserts having a plurality of rows and columns of medication storage slots defined therein. The smart medication cart includes a plurality of lights disposed along a top surface of each of a front edge and a side edge of each of the plurality of drawers, and configured to be placed in communication with a facility computing device capable of tracking a drawer and slot location in a cart insert held in that drawer at which resident medications can be found, the lights being configured such that, upon selection in the facility computing device of a medication to be administered to a resident, the facility computing device selectively illuminates only the lights on the drawer disposed on the front and side edges that are aligned with the specific medication slot row and column at the intersection of which is located the slot containing the selected medications.

According to certain aspects of the present disclosure a method of tracking medications to be administered is provided. The method includes assigning, in a pharmacy computing device, a medication of a resident to a desired slot in a cart insert having a plurality of slots defined therein arranged into a plurality of rows and columns. The method includes recording, in a database of a medication tracking service in communication with the pharmacy computing device, medication tracking data including a location of the assigned slot to which the medication was assigned in the cart insert, and an identification of the resident to whom the assigned medication belongs. The method includes allocating, in a facility computing device, the cart insert to a desired drawer position within a desired drawer of a medication cart. The method includes recording, in the database in communication with the facility computing device, additional medication tracking data, including identification of each of the specific drawer of the cart and the drawer position within the cart to which the cart insert is allocated. The method includes signaling the location at which the selected medication to be administered to the resident is located within the medical cart by: transmitting the medication tracking data from the database to the facility computing device, illuminating, by the facility computing device, a group of lights from each of the plurality of lights disposed along the font edge of the drawer that are visible when the drawer is in a closed position and a plurality of lights disposed along the side edge of the drawer, which illuminated group of lights are positioned at locations on the respective side and front edges of the drawer adjacent the respective row and column at which the slot is located in the cart insert within the drawer of the cart, at the intersection of which row and column the slot containing the medication is located.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF DRAWINGS

The present invention is described in detail below with reference to the attached drawing figures, wherein:

FIG. 1 is a schematic diagram of an embodiment of a system architecture according to certain aspects of the present disclosure;

FIG. 2 is a block diagram illustrating an exemplary embodiment of a pharmacy computing device, a medication shipping computing device, a facility computing device, and a medication tracking service from the architecture of FIG. 1, according to certain aspects of the present disclosure;

FIG. 3 is a isometric view of an exemplary embodiment of a smart medication cart, according to certain aspects of the present disclosure;

FIG. 4 is a top schematic view of an embodiment of a cart insert to be inserted into a drawer of the smart medication cart of FIG. 3;

FIG. 5 is a schematic isometric view of an embodiment of the smart medication cart of FIG. 3, schematically showing an embodiment of routing of wiring from the facility computing device to lighting along the sides and front of a drawer, and depicting one of the drawers of the cart in an opened position and a plurality of lights along the front and sides of the drawer illuminated so as to identify a slot from which medication is to be administered to a resident;

FIG. 6 is a schematic left side view of an embodiment of the smart medication cart of FIG. 3, schematically showing an embodiment of the routing of wiring from the facility computing device to lighting disposed along side edges of the drawers;

FIG. 7 is a schematic wiring diagram showing a schematic embodiment of routing of wiring to connect the facility computing device to lights to be affixed to a plurality of drawers of a medication cart according to certain aspects of the present disclosure;

FIG. 8 is an exemplary block diagram of a method according to certain aspects of the present disclosure;

FIGS. 9A-9G are example illustrations associated with the pharmacy computing device of the example process of FIG. 8, according to certain aspects of the present disclosure;

FIGS. 10A-10E are example illustrations associated with the facility computing device of the example process of FIG. 8, according to certain aspects of the present disclosure;

FIG. 11 is an example illustration of a slot assignment chart, according to certain aspects of the present disclosure;

FIG. 12 is block diagram illustrating an example computer system with which the example medication tracking service, the pharmacy computing device, the medication shipping computing device, and the facility computing device of FIG. 2 can be implemented;

FIGS. 13A-13G are top schematic views of various embodiments of a countertop of the smart medication cart.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

While the present invention is capable of being embodied in various forms, the description below of several embodiments is made with the understanding that the present disclosure is to be considered as an exemplification of the claimed subject matter, and is not intended to limit the appended claims to the specific embodiments described herein. The headings used throughout this disclosure are provided for convenience only and are not to be construed to limit the claims in any way. The various embodiments disclosed herein may be combined with other embodiments for the creation and description of yet additional embodiments.

Throughout the specification and claims, the following terms take the meanings explicitly associated herein, unless the context clearly dictates otherwise. The phrase “in one embodiment” does not necessarily refer to the same embodiment, although it may. Furthermore, the phrase “in another embodiment” or “in an alternate embodiment” does not necessarily refer to a different embodiment, although it may. Thus, as described below, various embodiments may be readily combined without departing from the scope or spirit of the present disclosure.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

The present disclosure is drawn to a computer-implemented method, apparatus, and system for filling, tracking, and administering medications from a smart medication cart to individuals in a facility, such as for example patients at a healthcare facility or hospital, or residents of a nursing home. While the present disclosure relates to the tracking and administering of medications to a plurality of individuals in any number and/or type of facilities and settings, in which facilities the individuals are either temporary or permanently residing (e.g. patients in a hospital, or residents in a nursing home), for sake of simplicity and consistency of terminology, the individual people to whom medication is prescribed or administered shall be referred to throughout this disclosure as a “resident” or “residents.”

### System Architecture

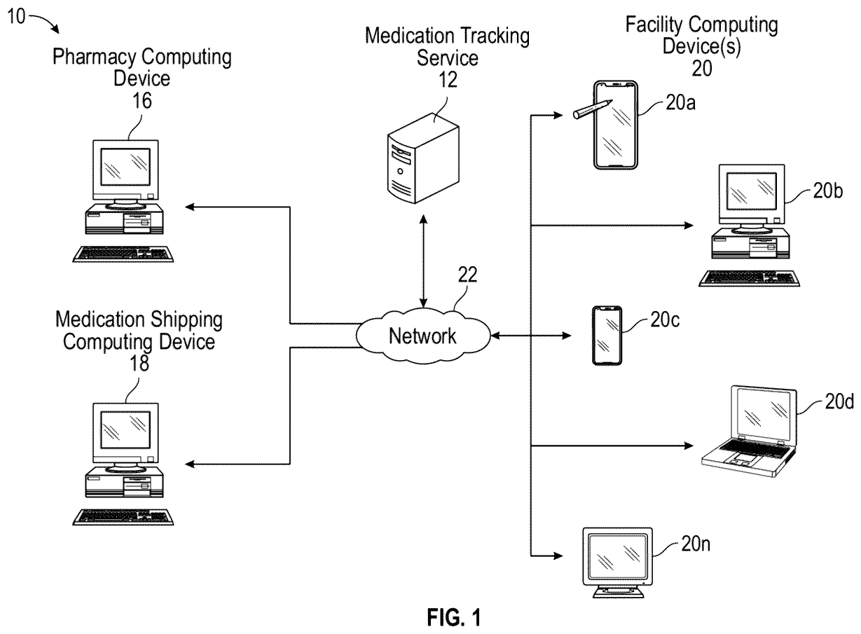

Referring to FIG. 1, an exemplary architecture 10 of a computerized system for filling and/or shipping prescriptions, tracking prescribed medications, and administering/dispensing medications is disclosed. The architecture includes at least one medication tracking service 12, a pharmacy computing device 16, a medication shipping computing device 18, and at least one facility computing device 20, all connected over a network 22.

The medication tracking service 12 can be any device having an appropriate processor, memory, and communications capability for communicating with one or more of the pharmacy computing device 16, the medication shipping computing device 18, and the at least one facility computing device 20 such as the first computing device 20a. For purposes of load balancing, the medication tracking service 12 may include multiple servers.

Each of the at least one facility computing device 20, such as the first facility computing device 20a, the pharmacy computing device 16, and the medication shipping computing device 18, with which the medication tracking service 12 can communicate over the network 22, can be, for example, a desktop computer, a tablet computer, a mobile phone, a mobile computer, a laptop computer, or any other device having appropriate input device, viewing screen or monitor, processor, memory, and communications capabilities. In certain aspects, the medication tracking service 12 can be a cloud computing server of an infrastructure-as-a-service (IaaS) and be able to support a platform-as-a-service (PaaS) and software-as-a-service (SaaS) services.

The network 22 can include, for example, any one or more of a personal area network (PAN), a local area network (LAN), a campus area network (CAN), a metropolitan area network (MAN), a wide area network (WAN), a broadband network (BBN), the Internet, and the like. Further, the network 22 can include, but is not limited to, any one or more of the following network topologies, including a bus network, a star network, a ring network, a mesh network, a star-bus network, tree or hierarchical network, and the like.

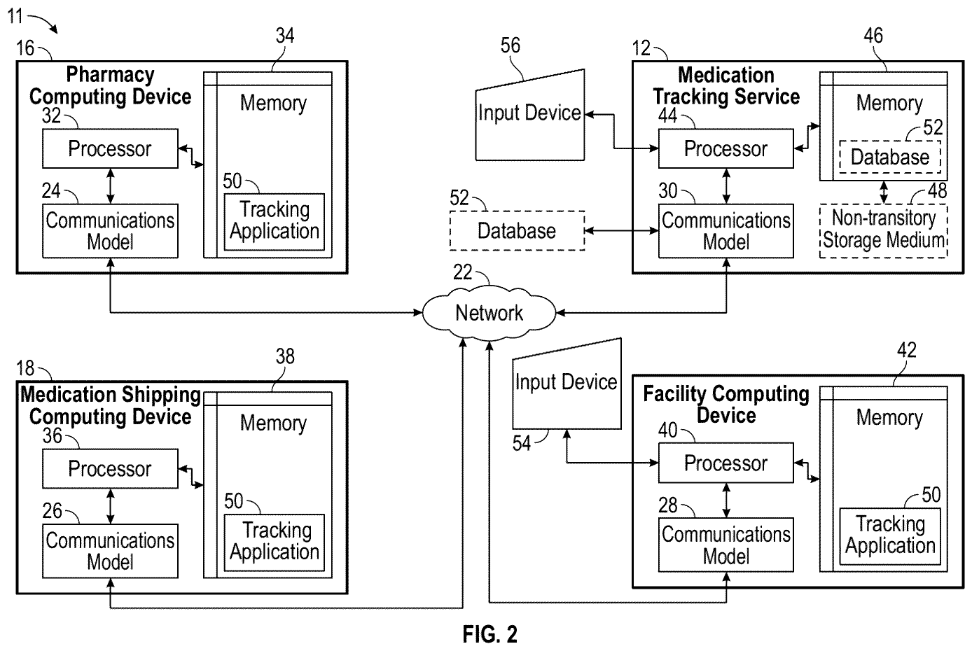

Referring to FIG. 2, a block diagram 11 illustrates examples of the pharmacy computing device 16, the medication shipping computing device 18, the at least one facility computing device 20, such as the first facility computing device 20a, and the medication tracking service 12 in the architecture of FIG. 1, according to certain aspects of the present disclosure. It should be understood that for purposes of explanation that the first facility computing device 20a is described, but any number of the at least one facility computing device 20 could be used in the system and method disclosed herein without departing from the scope of the present disclosure.

The pharmacy computing device 16, the medication shipping computing device 18, the at least one facility computing device 20, such as the first facility computing device 20a, and the medication tracking service 14 are each connected over the network 22 via respective communication modules 24, 26, 28, 30. The communication modules 24, 26, 28, 30 are configured to interface with the network 22 to send and receive information, such as data, requests, responses, and commands to other devices on the network 22. The communications modules 24, 26, 28, 30 can be, for example, modems or Ethernet cards.

The pharmacy computing device 16 includes at least one input device, a viewing screen or monitor, a processor 32, the communications module 24, and a memory 34. The processor 32 of the pharmacy computing device 16 is configured to execute instructions, such as instructions physically coded into the processor 32, instructions received from software in the memory 34, or a combination of both. The processor 32 of the pharmacy computing device 16 is configured to perform any of its functions described herein.

The medication shipping computing device 18 includes an input device, a viewing screen or monitor, a processor 36, the communications module 26, and a memory 38. The processor 36 of the medication shipping computing device 18 is configured to execute instructions, such as instructions physically coded into the processor 36, instructions received from software in the memory 38, or a combination of both. The processor 36 of the medication shipping computing device 18 is configured to perform any of its functions described herein.

The facility computing device 20 includes an input device, viewing screen or monitor, a processor 40, the communications module 28, and a memory 42. The processor 40 of the facility computing device 20 is configured to execute instructions, such as instructions physically coded into the processor 40, instructions received from software in the memory 42, or a combination of both. The processor 40 of the facility computing device 20 is configured to perform any of its functions described herein.

The medication tracking service 12 includes a processor 44, the communications module 30, a memory 46, and in some embodiments a non-transitory computer readable storage medium 48. One or more of the memory 46 or storage medium 48 may include a database programmed therein. The processor 44 of the medication tracking service 12 is configured to execute instructions, such as instructions physically coded into the processor 44, instructions received from software in the memory 46, or a combination of both. The processor 44 of the medication tracking service 12 is configured to perform any of its functions described herein.

### Smart Medication Cart

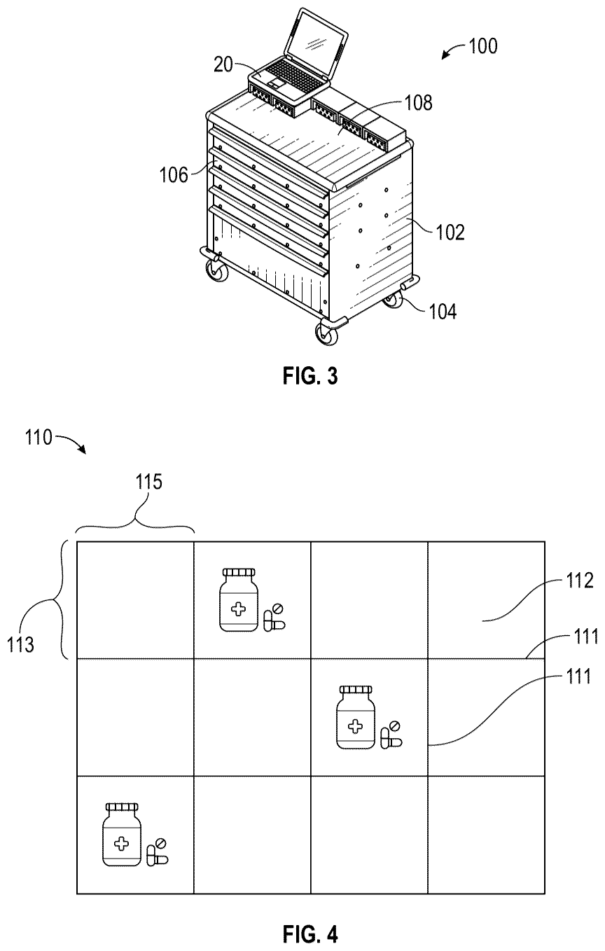

Referring to FIG. 3, an embodiment of a smart medication cart 100 for use in a method of storing, tracking, and administering medications to residents in a facility is disclosed. Generally speaking, a smart medication cart 100 of the present disclosure includes a cabinet 102 mounted on a plurality of wheels 104 or casters to permit the cart 100 to be moved from one location to another. The cart 100 further includes a plurality of drawers 106 disposed therein that slide in and out of the cabinet 102. In one embodiment the cart 100 may have drawers 106 that are all identical or uniform in size or dimension. In alternate embodiments, some of the drawers 106 in the cart 100 may be the same size or dimension while other drawers 106 in the same cart 100 may be a different size or have different dimensions without departing from the scope of the present disclosure. In one exemplary embodiment, as depicted in FIG. 3, the cart 100 may have five drawers 106 that are disposed successively in a single column with each drawer 106 extending across the entire width of the cabinet 102 between the cabinet walls, with each of the top four drawers having the same dimensions and the bottom drawer being taller in a height dimension (i.e. larger) than the top four drawers. In alternate embodiments, the cart 100 may have a different number of drawers 106, or different configuration or arrangement of such drawers 106 within the cabinet 102, with drawers disposed in a single column or alternatively in more than one column or row within the cabinet 102, without departing form the scope of the present disclosure.

The cart 100 further includes a countertop 108 working surface on which a facility computing device 20 is disposed. Each drawer 106 is configured to hold or accommodate therein one or more selectively removable cart inserts 110. Referring to FIG. 4, a cart insert 110 is a container for holding, storing, and/or transporting resident medications, that is configured to be inserted into and removed from a drawer 106 of the cart 100. Each cart insert 110 defines at least one slot 112 in which medications of residents are stored, and from which slots 112 the residents' medications can be removed to be administered to the residents by, for example, a nurse or other qualified medical or nursing practitioner. Each cart insert 110 may be subdivided by one or more dividing walls 111, into a number of rows 113 and columns 115, to define a plurality of individual slots 112 laid out in a grid pattern, in which slots 112 the medications of residents can be separated and stored. The medications of residents stored in each such slot 112 can also be removed therefrom to be administered to the residents by, for example, a nurse or other qualified medical or nursing practitioner. Each such slot 112 may contain one or more medications of only a single resident, such that any given individual slot is dedicated, at least temporarily, to that single resident, and the given slot contains only medications for that resident. In one embodiment, a drawer 106 may accommodate a single cart insert 110. In an alternate embodiment, a drawer 106 may be configured or dimensioned so as to accommodate two or more cart inserts 110 placed side-by-side, front-to-back, or a combination thereof.

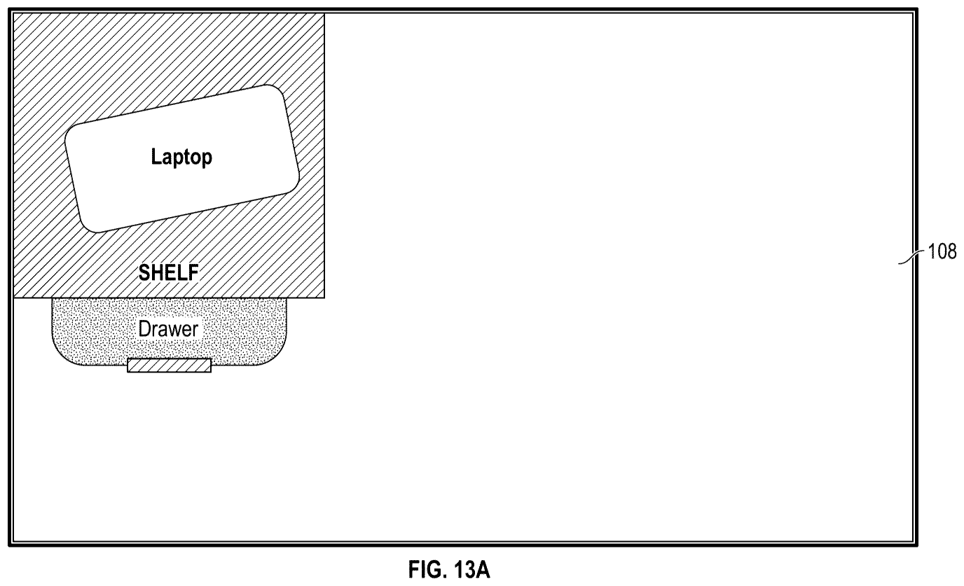

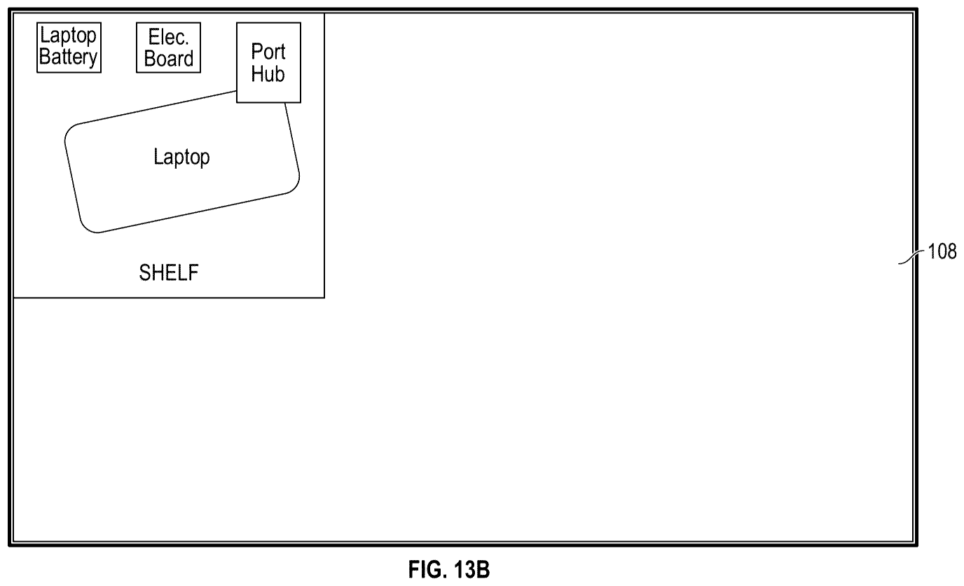

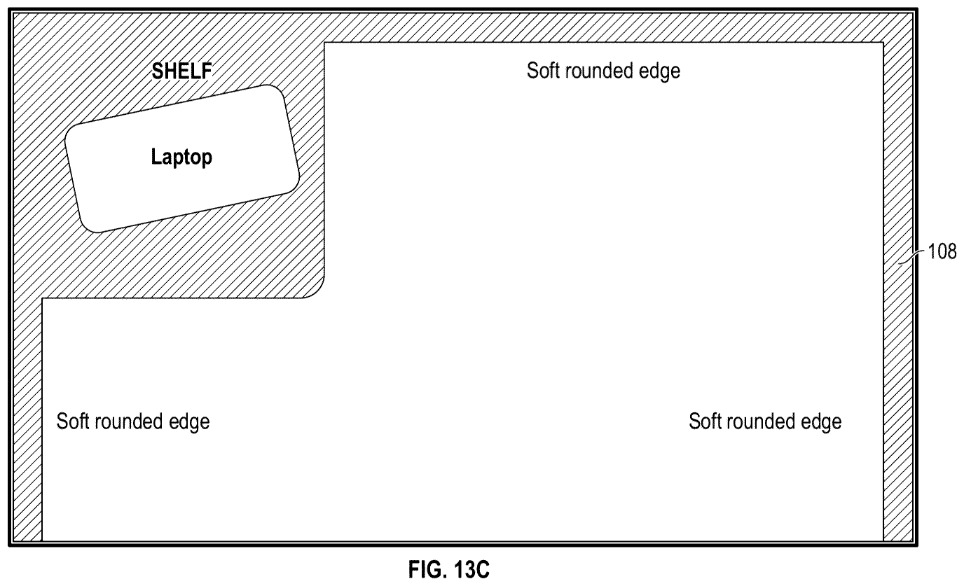

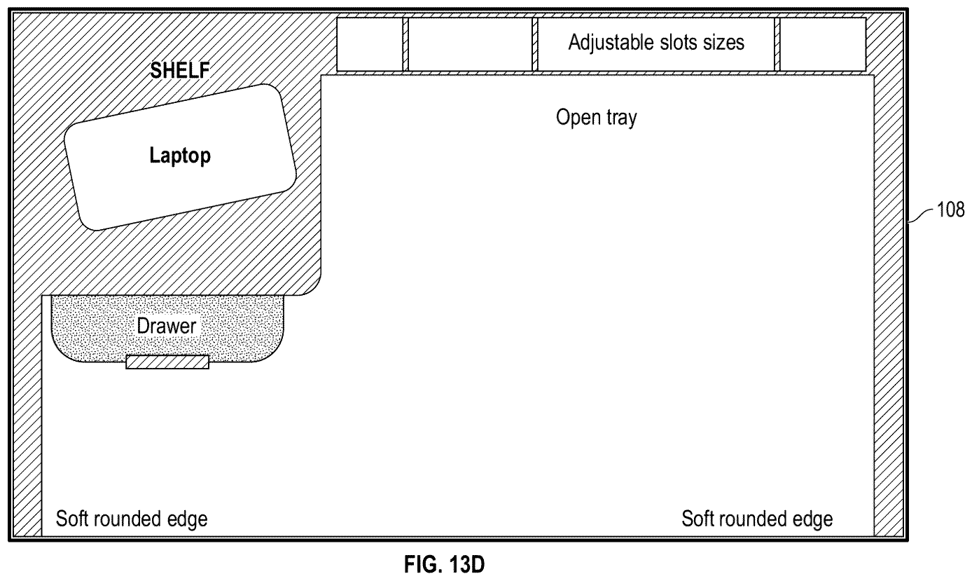

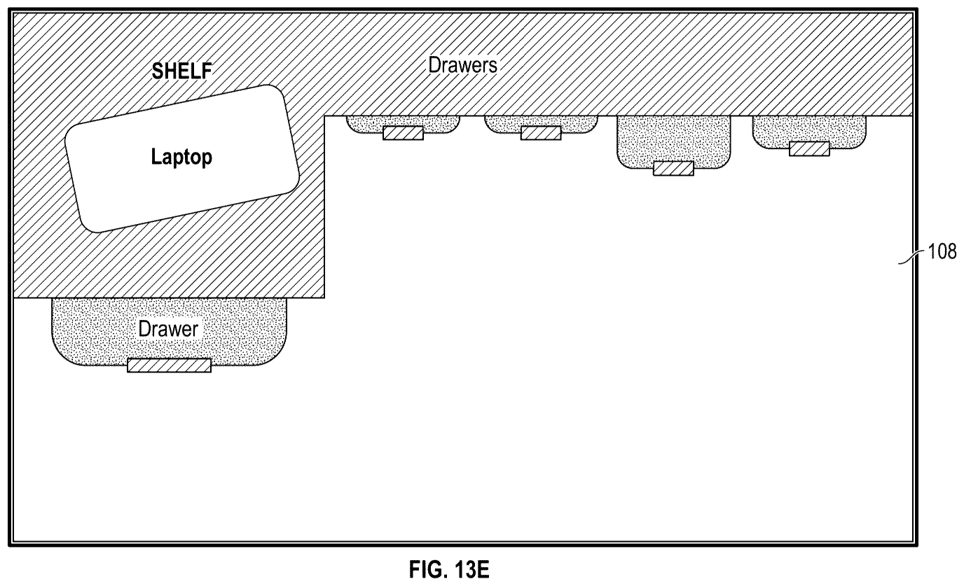

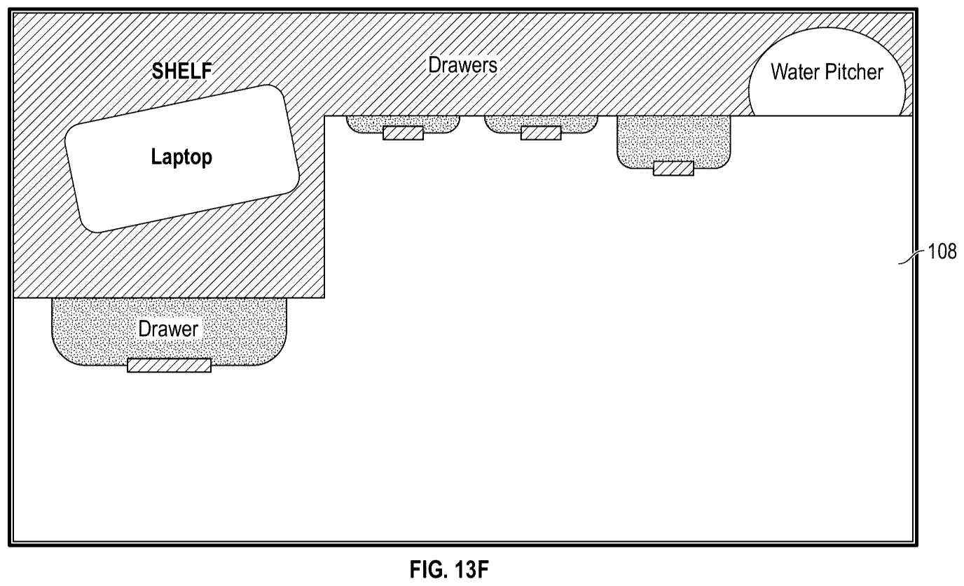

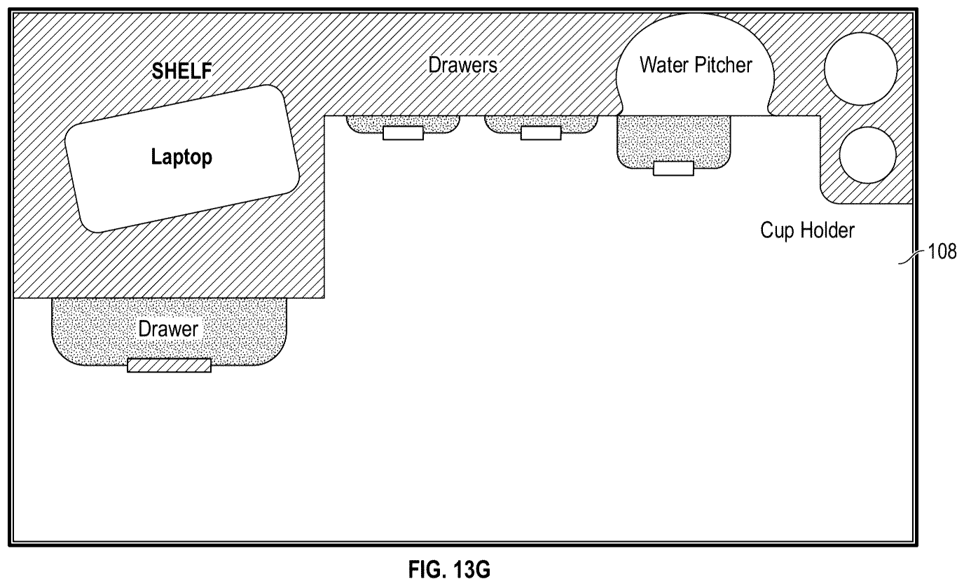

With reference to FIGS. 13A-13G, in various embodiments, the countertop 108 can have alternative configurations and arrangements of components including, but not limited to, shelves, drawers water pitcher recesses, cup holders, soft rounded edging, and other appropriate arrangements, sizes, and positioning of various components.

In one embodiment, the cart inserts 110 may be made out of cardboard, such that they are inexpensive to produce, reusable, and fully recyclable after they have become damaged or worn out from repeated use. In alternate embodiments, the cart inserts 110 may be made of plastic, metal or the like and as such may be much more durable. The slots 112 of the cart inserts 110 are configured to store medications of residents to be distributed by health care providers such as nurses in a hospital or nursing home, or other such qualified individuals.

Referring again to FIG. 4, in one embodiment a schematic top view of an exemplary cart insert 110 is shown that is divided, by a plurality of dividing walls 111, into three rows 113 and four columns 115 defining a total of twelve individual slots 112. In another embodiment (not shown) a cart insert 110 may have only a single slot 112 (i.e. it is not subdivided). In yet additional alternate embodiments (not shown), a cart insert may be divided into one row and two columns (i.e. a 1×2 cart insert) or two rows and one column (a 2×1 cart insert) with each arrangement of the cart insert 110 defining two total slots, two rows and two columns (i.e. a 2×2 cart insert) defining four total slots, two rows and three columns (i.e. a 2×3 cart insert) or three rows and two columns (i.e. a 3×2 cart insert) with each arrangement defining six total slots, three rows and three columns (i.e. a 3×3 cart insert) defining nine total slots, three rows and four columns (i.e. a 3×4 cart insert) or four rows and three columns (i.e. a 4×3 cart insert) with each arrangement defining twelve total slots, or any other such similar alternate division, of any number of rows (R) and columns (C) (i.e. a R×C cart insert) defining a number of slots 112 equal to R×C, without departing form the scope of the present disclosure.

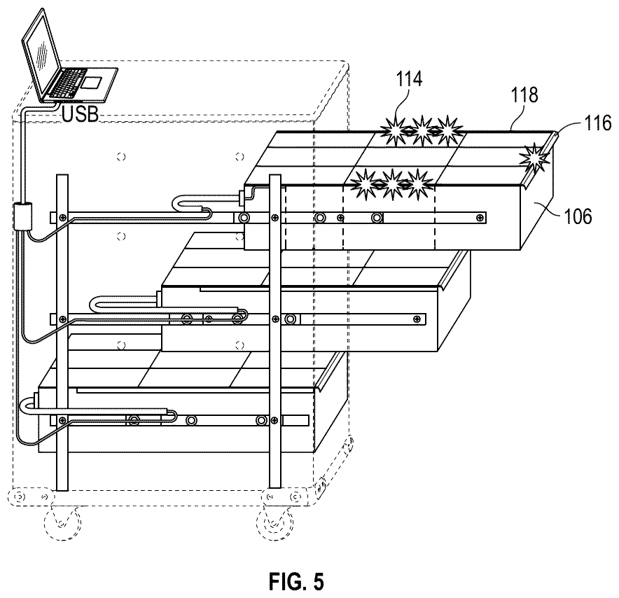

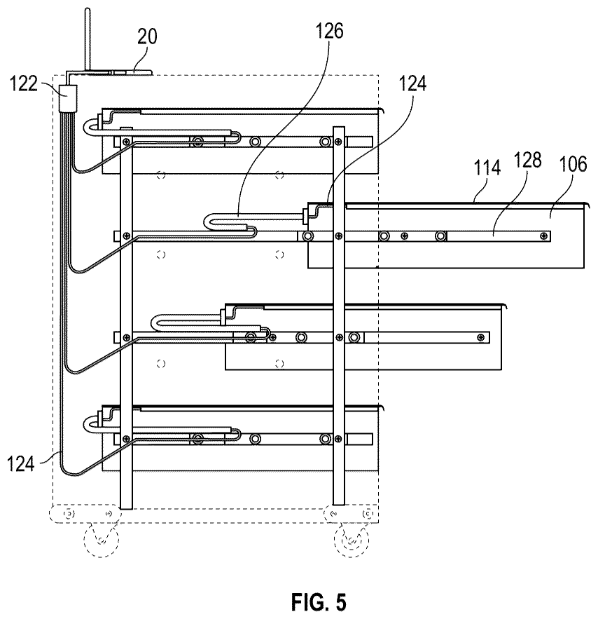

Referring to FIGS. 5-7, each drawer 106 includes a plurality of lights 114 disposed along each of a top surface of a front edge 116 and a top surface of at least one side edge 118 of the drawer 106. In alternate embodiments, the lights 114 are disposed along the front edge 116 and both opposing side edges 118 of one or more drawers 106. In still further alternate embodiments, the lights 114 are disposed along each of the front edge 116, a back edge 120 (not shown), and both opposing side edges 118 of one or more drawers 106. The lights 114 may be light emitting diode (LED) lights, in which a plurality of individually wired LED's are disposed on any one or more of such front, side, or back edges 116, 118, 120 of the drawers 106. In an alternate embodiment, the lights 114 may include strips of LED lights, in which each strip is configured to permit individual ones of the LED's on the strip, or a group of adjacent LED's on the strip, to be illuminated, turned on, or powered on, while the remaining LED's on the strip remain turned off or unlit. In still alternate embodiments there may be multiple strips of LED lights disposed end to end on a given front, side, or back edge 116, 118, 120 of the drawer 106 without departing from the scope of the present disclosure. In one embodiment, the lights 114 disposed on the front edge 116 of the drawer 106 are visible at all times, regardless of whether the drawer 106 is opened or closed.

The lights 114 are wired into, and/or in electrical communication with, a light control module 122. The light control module 122 may be an Arduino processor that is reconfigurable (e.g., a control processor board), or other such similar control module capable of controlling the selective on/off functionality of the lights 114. The control module 122 is in communication with, controlled by, and powered by the facility computing device 20. In one embodiment the facility computing device 20 is a laptop computer that controls, powers, and sends lighting command input signals to the control module 122 via a USB cable connected between the control module 122 and the facility computing device 20. In such embodiments, the facility computing device 20 is also used to provide the electrical power, such as from a rechargeable laptop battery, to the lights 114 through the control module 122. The control module 122 turns the lights 114 either on or off in response to control signals it receives from the facility computing device 20.

Any communications or power cabling/wiring 124 connecting any of the facility computing device 20, the control unit 122, or the lights 114 may be hidden within the cabinet 102, between the walls of the cabinet 102 and outer surfaces of the drawers 106 disposed therein, or under a protective shelf or covering. The cabinet 102 of the cart 100 may include a removable rear maintenance panel to provide access to any hidden wiring 124 disposed between the inner surfaces of the walls of the cabinet 102 and the outer surfaces of the drawers 106 disposed within the cabinet 102. Wiring 124 within the cabinet 102 of the cart 100 running from the control module 122 to the lights 114 may be routed at least partially in cable or wire carriers 126 that run along, or substantially parallel to, at least one of the drawer glides 128 associated with each drawer 106. The drawer glides 128 permit the drawers to be opened and closed. The wire carriers 126 disposed adjacent thereto protect the control wiring 124 for the lights 114 and permit the wiring 124 to travel back and forth with the drawer 106 when the drawer 106 is opened and closed, without any risk that the wiring 124 will get caught in the drawer glides 128 where it could be damaged or severed resulting in a short circuit or loss of connection to the lights 114.

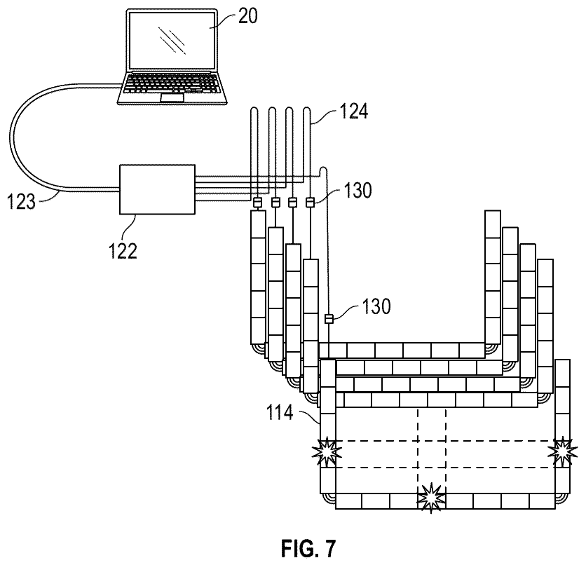

Referring to FIG. 7, in certain embodiments, quick disconnect electrical connectors 130 are disposed at one or more points along the wiring 124 between the lights 114 and the control module 122, thereby making it easy to disconnect and replace one or more lights 114, or strips of lights 114, that may become damaged during use or fail over time. For example, a quick disconnect electrical connector 130 may be disposed at a rear corner of a drawer 106 to allow for easy disconnection, removal, and replacement of a faulty or failed light 114, from either the front of the cart 100 when a drawer 106 is pulled forward to its open position, or through the removable back panel (not shown) where all of the wiring 124 is accessible. The lights 114 on the drawers are programable via the facility computing device 20 and the control module 122, such that a user may trigger the illumination (i.e. turning on) of only the lights 114 aligned with any given row 113 and column 115 that corresponds to a selected slot 112 in the cart insert 110. In certain embodiments, the strip of lights 114 can be disposed within the framework of the drawer 106. In certain embodiments, each strip of the lights 114 can be replaced independently of each other

In certain embodiments, the medication cart 100 is configured such that when a nurse needs to make the rounds of the facility with the medication cart 100 to administer medications to one or more residents, the nurse may, place a facility computing device 20 on the countertop 108 of the cart 100 and connect a USB cable 123 from the light control module 122 to a USB port of the facility computing device 20. The nurse then logs into a medication tracking application 50 on the facility computing device 20, and navigates to a medication administering tab or page. The nurse then either uses a mouse, stylus or finger on a touch screen, a keyboard, or other such GUI input device of the facility computing device 20, to select a resident to whom medications are to be administered from a list of residents stored in a database 52 and displayed onscreen in a GUI of the tracking application 50.

Selecting a resident in the tracking application 50 initiates the tracking application 50 to recall from the database 52 and display onscreen a list of the specific medications currently stored in the cart 100 that are to be administered to the resident. The tracking application 50 also recalls from the database 52 the specific drawer 106, and the specific slot(s) 112 within the drawer 106, at which the medications are being stored. Upon selection of a medication by the nurse from the list displayed onscreen, for example, the tracking application 50 triggers the facility computing device 20 to send a lighting control signal to the light control module 122, to illuminate a subset of lights 114 on the drawer 106 (shown in FIG. 5) in which the selected medication is located. With reference to FIG. 4, the subset of lights 114 that are illuminated are those that are located adjacent the specific row 113 of slots 112 and column 115 of slots 112 that are aligned with the slot 112 of the cart insert 110 disposed within the drawer 106 in which the resident's medications are being stored.

The lights 114 along the front edge of the drawer 106 are visible to the nurse even when the cart is closed, such that the nurse can first easily see which drawer has lights 114 that are turned on or illuminated, and therefore identify and open the specific drawer 106 in which the resident's medications are stored. Once the nurse opens that specific drawer 106, the nurse is also then able to clearly see the subset of illuminated lights 114 on the front edges and on both side edges of the drawer 106. The only lights 114 that are illuminated on the front and side edges of the drawer 106 are those lights 114 that are adjacent to the specific row and specific column of the cart insert 110 that are aligned with, and correspond to, the specific slot 112 at which the resident's medication are stored. In this manner, the slot 112 found at the intersection of the lighted row 113 and the lighted column 115 is where the resident's medication to be administered can be found, thus providing the nurse with an easy visual indication of the location at which the medications are stored. From there the nurse can quickly administer the appropriate prescribed medications form that slot 112, and return any unused part of a prescription back to the same slot.

### Method

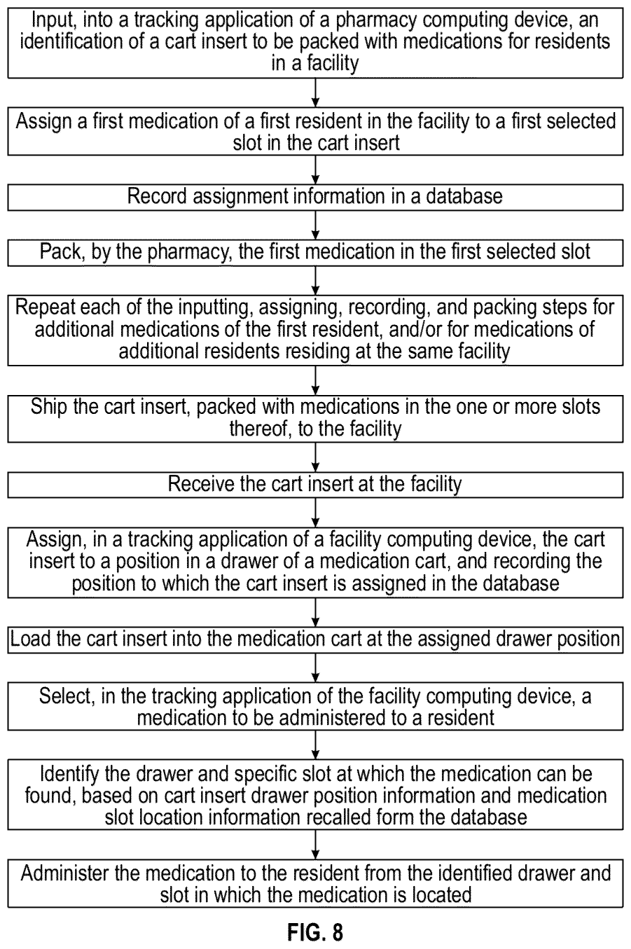

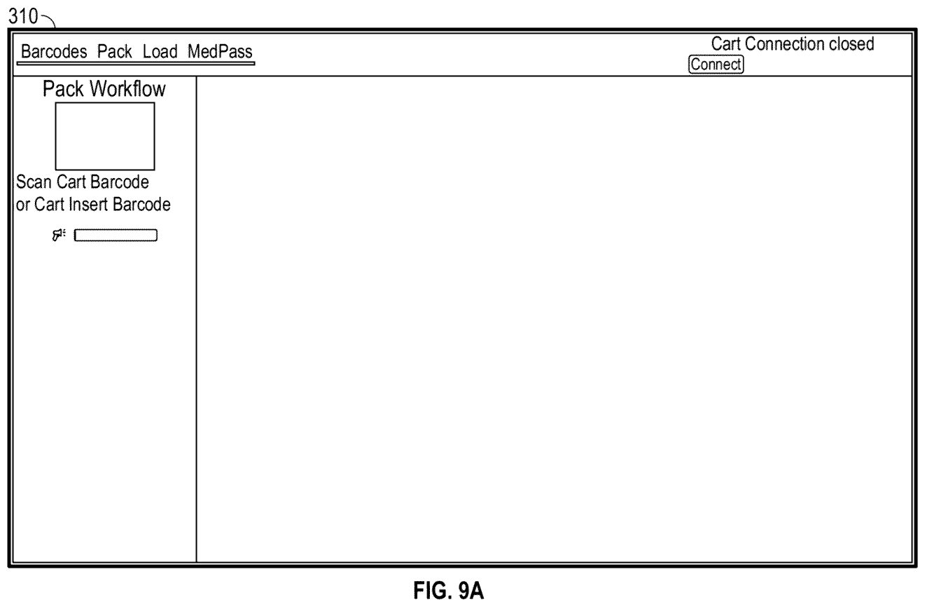

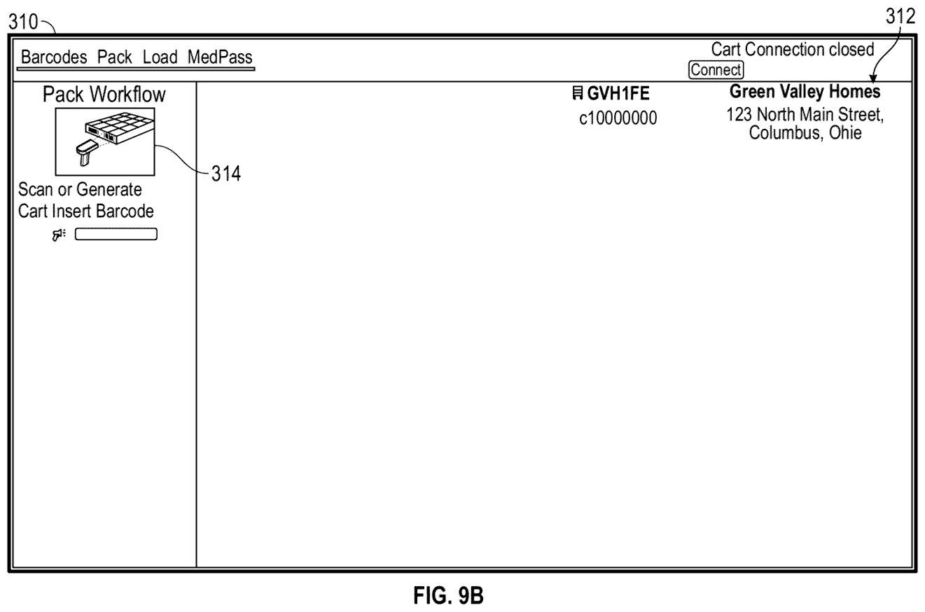

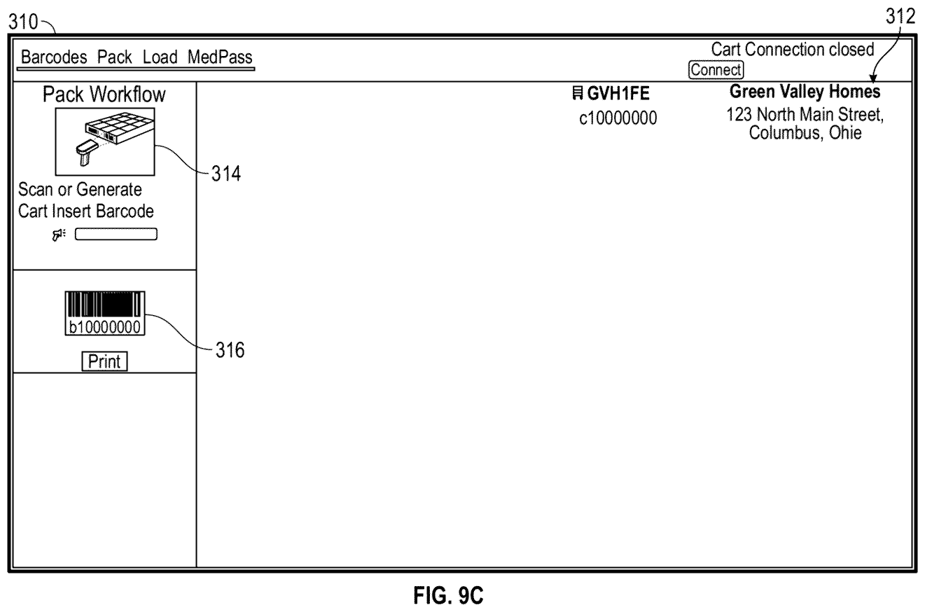

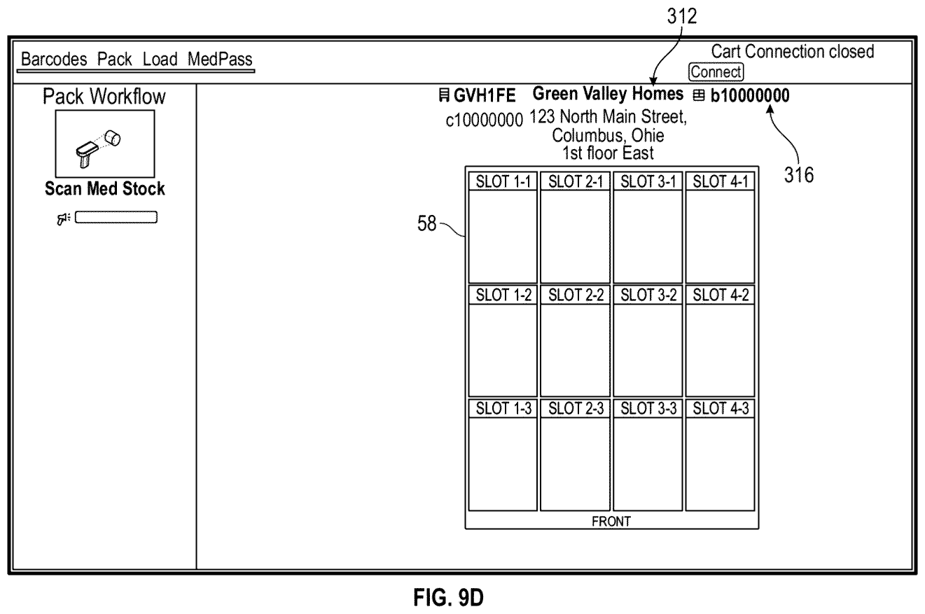

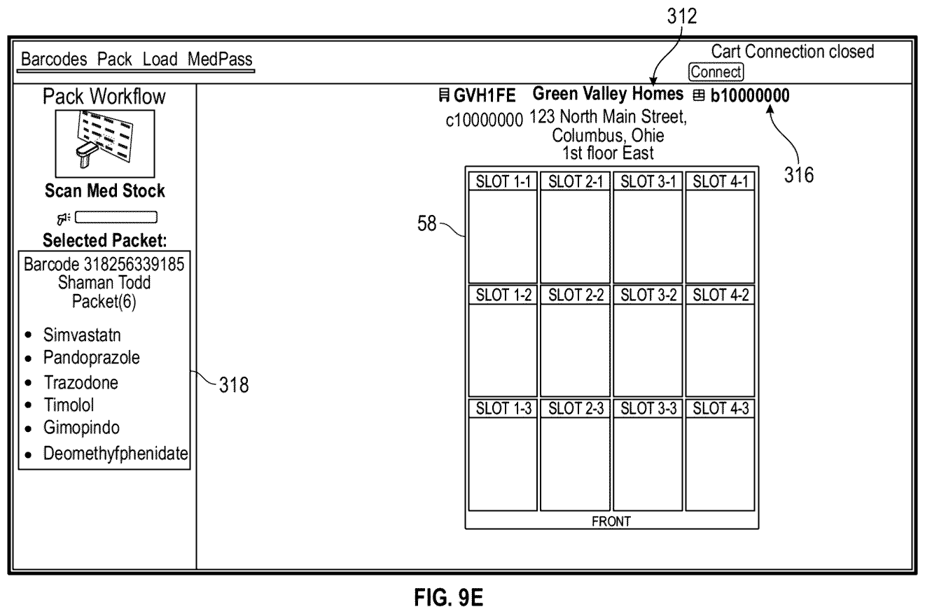

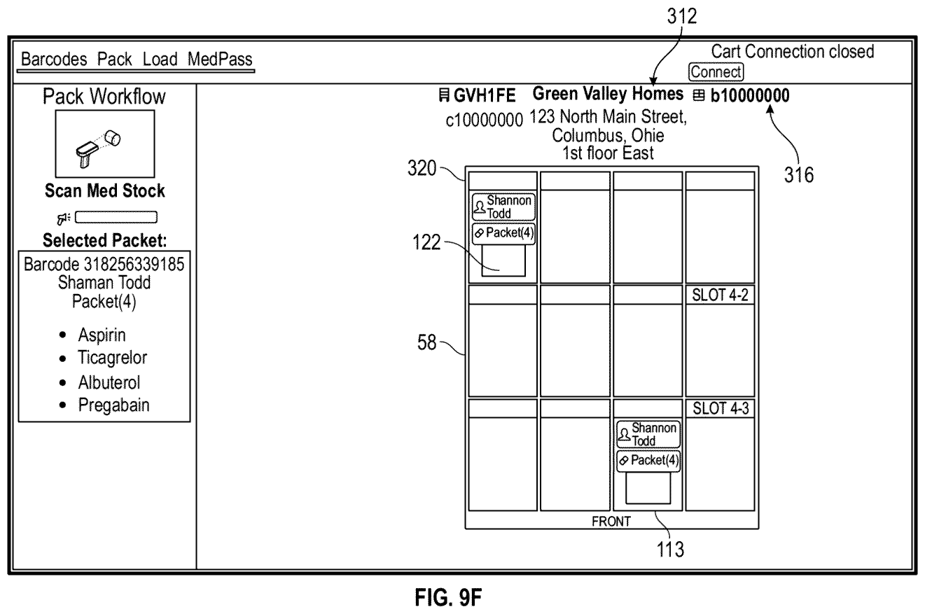

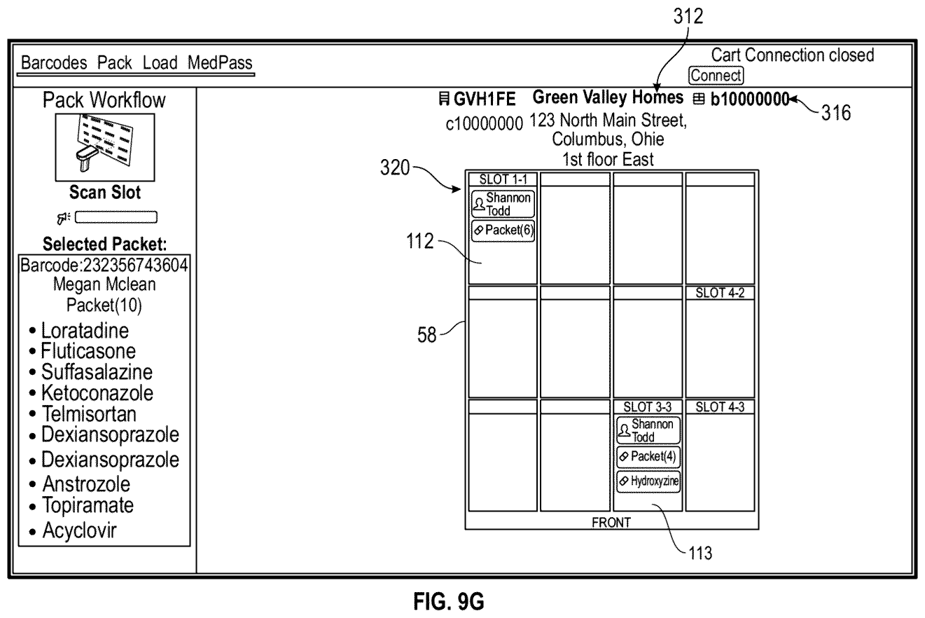

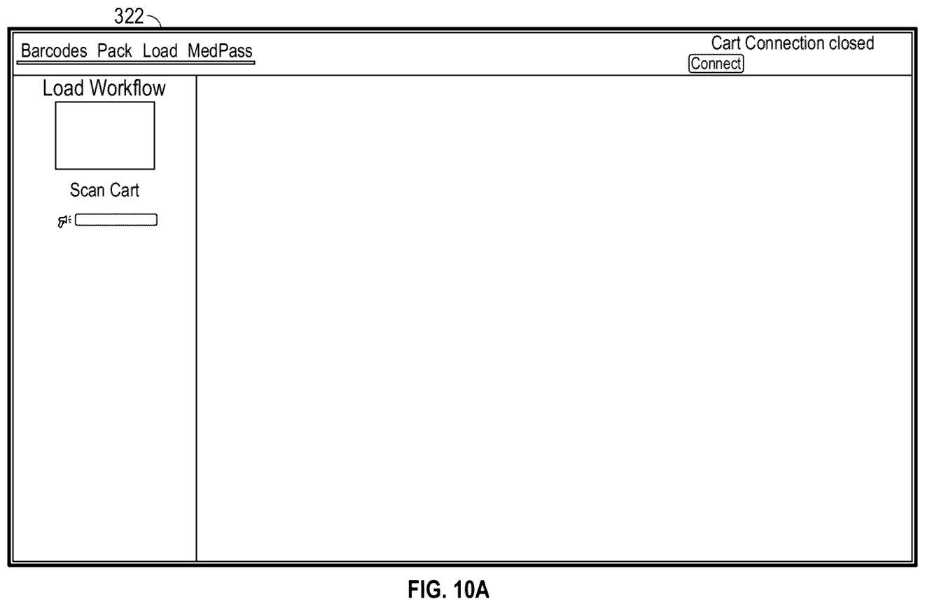

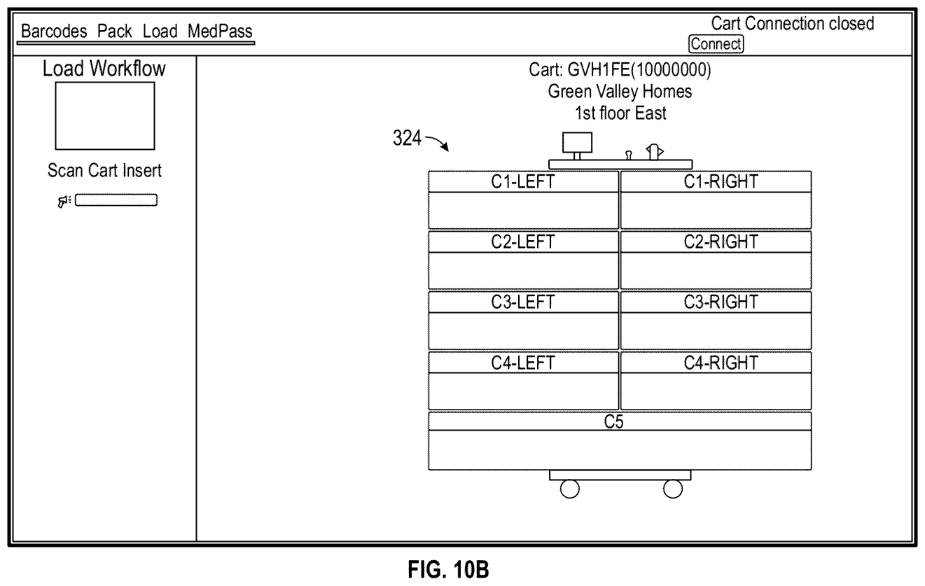

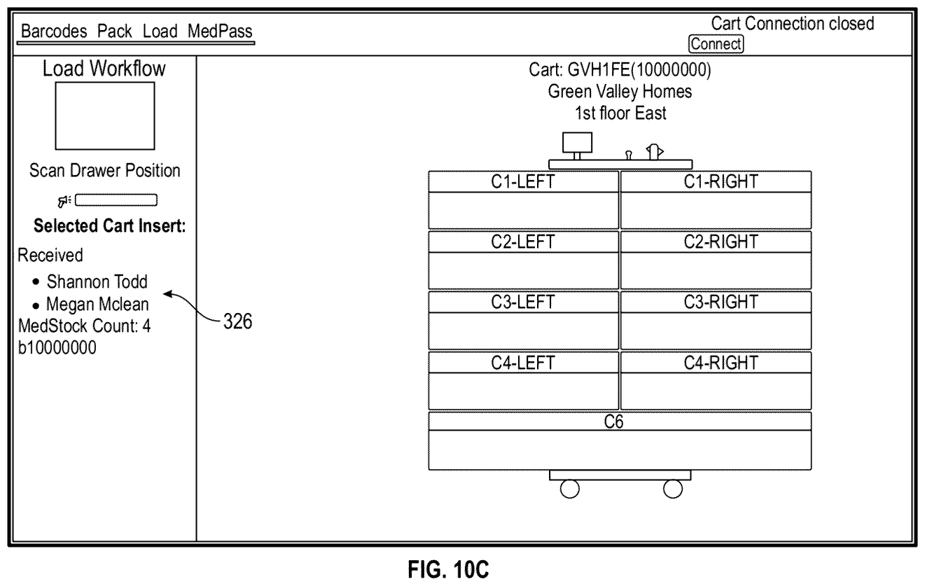

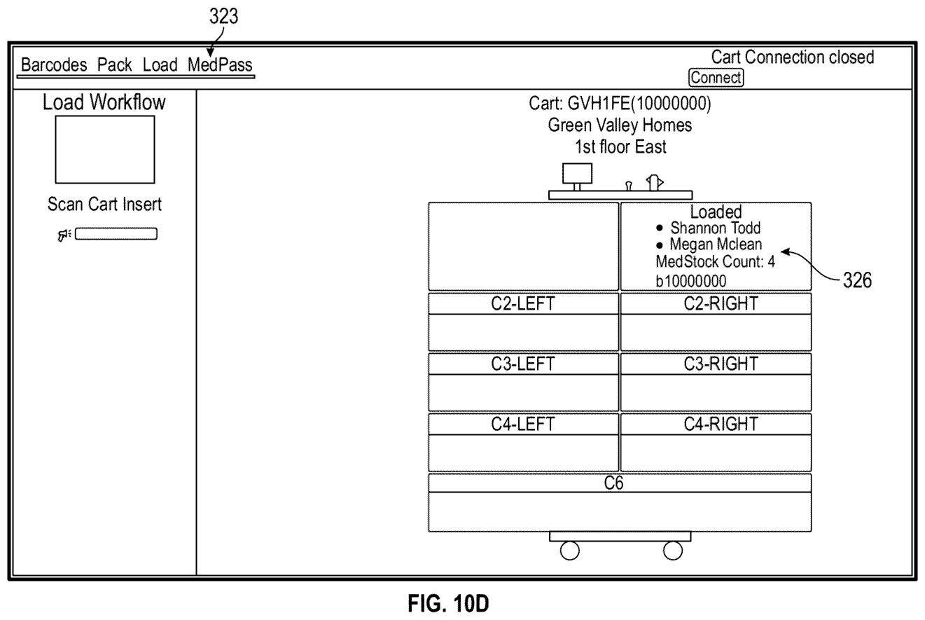

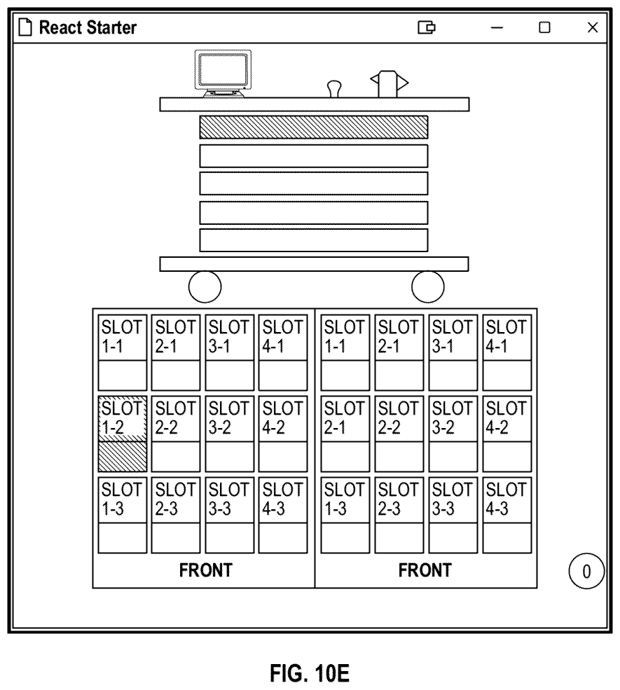

Referring generally to FIG. 8, in full operation, the complete system from prescription filling to administering of the medications operates according to the example process of FIG. 8 and will be described with reference to example screenshots shown in FIGS. 9A-9G and 10A-10E. In certain embodiments, a computer-implemented method of the present disclosure includes receiving by the pharmacy computing device 16, one or more prescriptions for medications for one or more residents at a remote facility, filling the prescription, and preparing the filled prescriptions for shipping to the facility. Such prescriptions are first filled at the pharmacy by a pharmacist or pharmacy technician. A label containing a unique medication identifier, such as, but not limited to, a barcode, a QR code, and other appropriate identifiers, is printed and applied to the packaging of the medication. In certain aspects, the label is printed at a printer via instructions from the pharmacy computing device 16. The packaging can be a pill bottle, medication tear strips, blister packaging, bottle, bag, or other such similar packaging for medication. The label may contain additional information such as resident name, medication name, and/or any dosing instructions. The medication barcode on the medication packaging is associated with the patient name, medication type, dosing instructions, and or any other pertinent information and can be used to record such information into a database for later recall upon the scanning of the medication barcode by a scanner in communication with a computing device.

With particular reference to FIGS. 9A-9G, functionality of the pharmacy computing device will be described in detail. In certain aspects, the method continues by the pharmacist or technician logging into a medication tracking application 50 on the pharmacy computing device 16. The medication tracking application 50 is in data communication, via the network 22, with the medication tracking service 12 and a medication tracking database 52 programmed into one or more of the memory 46 or a non-transitory computer readable storage medium 48 of the medication tracking service 12. In certain other embodiments, the medication tracking database 52 may be a separate database in communication with the medication tracking service 12. The medication tracking application 50 can facilitate displaying a graphical user interface (GUI) on screen of the pharmacy computing device 16. The pharmacy computing device 16 further includes, or is in communication with, an input device 56, such as for example a scanner, which may be a handheld scanner, a barcode scanner, a flatbed scanner, a tablet or smart phone using a camera with appropriate scanning software, or other such similar scanner for scanning barcodes.

The pharmacy prepares one of a plurality of available empty cart inserts 110 to be packed by the pharmacy with medications from prescriptions that are for residents who all reside at a single facility, or more specifically, at one internal location within a given facility. An internal location is a unique location within a given facility, such as for example, the 1^{st} Floor West Wing, of a nursing home. All of the medications to be packed into a cart insert 110 will be for residents that all reside at the facility, or at a specific internal location within the facility, and to which facility the cart insert 110 will be shipped.

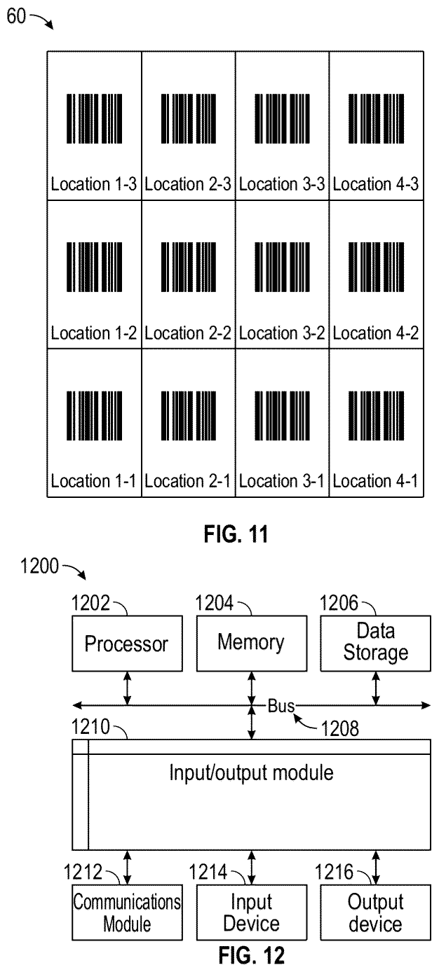

The technician prepares to pack the medications of the filled prescriptions into a cart insert 110 for a given facility or internal location, by clicking on a pack tab 310 to open or navigate to a packing tab, page, function, or workflow in the onscreen GUI of the tracking application 50. In certain embodiments of the present system and methods, inputs to the system are based primarily on the scanning of unique identifiers, such as, but not limited to, barcodes, QR codes, and other appropriate identifiers, throughout the process. In such embodiment, the pharmacy technician has a master manual for the system listing out various facilities, and/or, depending on the size of the facility, numerous internal locations within each facility. Each facility and/or internal location 312 listed in the manual has its own unique location barcode printed in the manual adjacent the name of the facility or internal location, that is used to identify the facility or internal location in the tracking application 50. The technician scans a location barcode in the master manual corresponding to a first internal location of a facility, which tells the tracking application 50 that a cart insert and the medications to be subsequently packed therein (see below with reference to FIG. 11) is destined for residents residing at that internal location, which is then displayed on the pharmacy computing device 16, as illustrated in FIG. 9B. The tracking application 50 on the pharmacy computing device 16 then displays a prompt 314 for the technician to either scan an existing cart barcode label previously applied to an empty cart insert 110, or to print out a new cart identification barcode label 316, as representatively displayed on the pharmacy computing device 16, and apply it to the empty cart insert 110. This cart barcode label for the cart insert 110 permits the tracking application 50 to identify and record that specific cart insert 110 and ultimately the medications packed therein, in the tracking application 50. As depicted in FIG. 9D, the technician next scans the printed cart barcode applied to the cart insert 110, which then triggers the tracking application 50 to record in the database 52 of the medication tracking service 12 that this specific identified cart insert 110, and any medications that are to be assigned and packed into slots 112 thereof, are to be shipped to the internal location 312 of a facility for which the cart insert was scanned. Scanning the cart barcode also initiates a loading workflow in the tracking application 50 for the cart insert 110 that is ready to be loaded with medications. Simultaneously, as depicted in FIG. 9D, the tracking application 50 generates and displays an onscreen graphic representation 58 of an empty cart insert having the same number and arrangement of virtual slots as the real, corresponding cart insert 110.

The technician next scans the medication barcode label applied to a first medication package of a resident's filled prescription. Scanning the medication barcode triggers the tracking application 50 to retrieve from the database 52 of the medication tracking service 12 resident information 318, such as, but not limited to, the name of the resident to whom the medication was prescribed and the pertinent medication information, and display it onscreen in the tracking application 50 on the pharmacy computing device 16, as depicted in FIG. 9E.

Referring to FIG. 9F, during the package process, the technician next electronically assigns, marries, or maps the scanned medication to an empty slot 112 in the cart insert 110, and the tracking application 50 records in the database 52 of the medication tracking service 12 such assignment or mapping for later recall. In one embodiment, to electronically assign the scanned medication to an empty slot 112 of the cart insert 110, the technician has available to him for scanning a slot assignment chart 60 (see FIG. 11), which chart may be wall mounted, table mounted, or a page in the master manual in the possession of the technician. The chart 60 is a grid formed from a plurality of adjacent squares that are representative of the each of the slots 112 in the cart insert 110, and that are arranged into the same number of rows and columns as the slots 112 in the real cart insert 110 to be packed by the technician. Each square on the chart has its own slot position unique identifier, such as, but not limited to, a barcode, a QR code, and other appropriate identifier, that respectively corresponds or maps to each of the respective positions of each slot 112 in the prior scanned cart insert 110 to be packed by the technician, and also corresponds to a graphic representation of that slot in the onscreen graphic representation 58 of the cart insert that is displayed on the pharmacy computing device 16 via the tracking application 50. In certain aspects, the technician uses a handheld scanner, such as the input device 56, to scan one of the slot position barcodes printed on one of the squares on the chart 60, which then assigns the previously scanned medication to the empty slot 112 corresponding to the square of the chart that was just scanned.

As also depicted in FIG. 9F, upon assignment of the medication to an empty slot 112 in the cart insert 110, the tracking application 50 on the pharmacy computing device 16 also then places that resident's name and scanned medication information 320 into the corresponding slot position of the onscreen graphical representation 58 of the cart insert displayed in the tacking application 50. The onscreen graphic representation 58 of the cart insert 110 provides a visual aid to the technician during the assignment and packing of medications to the cart inserts 110, and permits the technician to visually see or confirm the slot position in the cart insert 110 to which the medication has been assigned and into which each medication must be packed by the technician.

The technician then packs, loads, or places the scanned medication into the corresponding slot 112 of the actual cart insert 110 into which such medication was just assigned. The medication tracking database 52 of the medication tracking service 12 then stores the assignment and/or mapped information, specifically, one or more of the name of the resident, the name of the medication, the medication information, and the location of the slot 112 within the cart insert 110 to which the medication has been assigned, so that such information can be recalled therefrom at a later time.

While certain embodiments disclose the scanning of a slot position barcode on an assignment chart to assign the medication to a slot 112, in alternate embodiments, after the medication barcode for the medication has been scanned, the medication may be assigned to a specific slot 112 in the cart insert 110 by selecting, via a touch input on a touch screen or selection by a computer mouse or other similar input device, one of the empty slots in the onscreen graphic representation 58 of the cart insert. Such selection in the tracking application alone may complete the assignment, thereby mapping that resident's name and medication to the corresponding slot 112, and the technician may then load the actual medication into the corresponding slot 112 in the cart insert 110.

The process of selecting or scanning resident medication to be shipped to a given internal location, assigning the medication to various slots 112 in the cart insert 110 to be shipped to the internal location, and loading the medication into the assigned slot 112 of the cart insert 110 can be repeated for each additional medication prescribed to the resident, or for additional residents whose medications are to be shipped to the same internal location, until all such slots have been filled. The process can then be repeated with a new cart insert 110 for additional medications and residents.

Each slot into which a medication is assigned is associated with a single specific resident, rather than to a specific bed in a multi-resident room, or to a given room or apartment. In this regard, since each slot is assigned only to a single resident, if there is still space available in a given slot 112 of the cart insert 110 after assigning a first of the resident's medications thereto, and the resident has more than one medication, the tracking application 50 will permit the technician to assign additional of the resident's medications to the same slot 112 and load such additional medication in the same actual slot 112. Otherwise, if the slot 112 is full, the technician can load the resident's additional medication to a different empty slot 113, for example, in the same cart insert 110. If the technician scans the medication of a second resident for packing in the cart insert 110, the tracking application 50 will gray-out and/or otherwise indicate as “occupied” any slots 112 in the onscreen graphical representation of the cart insert that have already been assigned to medications of other residents, as illustrated in FIG. 9G. If the technician tries to assign the medications of the second resident to a slot 112 that has already been assigned to medications of another resident, the tracking application 50 will not permit such assignment to be completed. Only the remaining empty slots, or slots previously assigned to medications for that second resident, will be available to assign further medications for the second residents. If the technician attempts to assign to a slot 112 of the cart insert 110 any medications for a resident who no longer resides at the internal location to which the cart insert is to be shipped, the tracking application will gray out any empty slots and/or otherwise indicate that they are unavailable, and prevent such medications from being assigned to any slot of that cart insert 110. In such situations, the tracking application 50 may also display a message that such resident does not reside at the internal location to which the cart insert 110 will be shipped. It should be understood that when a single patient or resident's medication is loaded into two different slots, during disbursement of the medication, the nurse will administer one medication slot at a time.

The steps of scanning a resident's medication, assigning the medication to a slot 112 of the cart insert 110 via the tracking application, and loading the medication into the slot 112 of the cart insert 110 is repeated for each such resident who has medication to be assigned and loaded into a cart insert 110 for shipping to an internal location of a facility, until all of the slots 112 of the cart insert 110 have been filled or until the technician has no further medications to ship to a given internal location. At this point, the cart insert 110 is ready to be shipped to the designated internal location of the facility that was selected by the technician at the beginning of the loading workflow.

Once the cart inserts 110 are ready to be shipped, a medication shipping computing device 18 (which may be the same computing device, or a different computing device than the pharmacy computing device 16), may be used to ship the cart inserts 110 loaded with medications. A tote barcode of a shipping tote may be scanned by an input scanner of the medication shipping computing device 18. The cart insert barcodes of each cart insert 110 that is to be packed into the shipping tote and sent to a single internal location of a facility may then also be scanned by an input scanner of the medication shipping computing device 18. Scanning the cart insert 110 recalls from the database 52 of the medication tracking service 12 the internal location and facility to which the medications within the cart insert are to be shipped, and uses that information to either prepare a shipping label for the shipping tote, or inform the shipping company as to the shipping address. Only those cart inserts 110 having medications assigned to the same internal location are permitted by the tracking application 50 to be packed into the same shipping tote. If the cart insert barcode of a first cart insert 110 going to a first internal location of a facility is scanned and packed into a shipping tote (thereby assigning the shipping address of that tote to the first internal location), and the cart insert barcode of a second cart insert 110 that is to go to a second internal location or facility is thereafter scanned for that same tote, the tracking application 50 will display an error or warning message indicating to the shipper that second cart insert is to be sent to a different location than the first cart insert and the second cart insert cannot be assigned to that specific tote.

The scanning of the totes and the cart inserts 110 packed therein creates a record in the database 52 of the medication tracking service 12 as to the specific tote that is being shipped out from the pharmacy, the location of the facility and/or its internal location to which the tote is being shipped, the specific cart inserts 110 contained within the tote being shipped, the specific medications that are packed into each such cart insert 110, the specific residents to which each such medication is prescribed, and the slot locations assigned to each such resident and their medications contained in each such slot 112. In this regard, the tracking application 50 and the medication tracking service 12 fully document and track all of the packed and shipped medications as they leave the pharmacy in totes and are shipped to the destination facility and/or internal location.

In certain aspects, the method continues by receiving in one of the facility and/or an internal location thereof, a tote shipped from the pharmacy. The same medication tracking application 50 is opened on a facility computing device 20. The tracking application 50 on each such facility computing device 20 is connected via the network 22 to the medication tracking service 12 and the medication tracking database 52, so as to enable each facility computing device 20 and the tracking application 50 loaded thereon to retrieve stored data from the database 52 of the medication tracking service 12. An input device 54, for example a scanner, such as, a handheld barcode scanner similar to that used by the pharmacy, is used to scan the tote identification barcode on the tote received from the pharmacy. This scan will indicate, for facilities that have different smart medication carts 100 servicing different internal locations within the facility, the specific internal location within the facility to which each received tote, and the cart inserts 110 contained therein, should be sent for unpacking into the smart carts 100 servicing only that specific internal location. Once the tote arrives at the designated internal location within the facility, the cart inserts 110 therein are unpacked and ready to be loaded into a medication cart 100 servicing that internal location.

With particular reference to FIGS. 10A-10E, functionality of the facility computing device 20 will be described in detail. In certain aspects, the method further continues by a nurse or other authorized individual retrieving one of the cart inserts 110 received from the pharmacy. As depicted in FIG. 10A, the nurse logs into the tracking application 50 on a facility computing device 20 assigned to the medication cart 100 for that internal location, and clicks on a load tab 322 to open or navigate to a load page, tab, function, or workflow of the application 50 to initiate the loading workflow for the cart 100. The tracking application 50 of the facility computing device 20 is in communication, via the network 22, with the medication tracking service 12 and the medication tracking database 52, which may be separate or stored therein. The nurse utilizes the input device 54, such as the handheld scanner, of a facility computing device 20 to first scan a cart unique identifier, such as an identification barcode, affixed to the medication cart 100, thereby identifying for the application 50 the specific medication cart 100 about to be loaded with the newly received cart inserts 110. As depicted in FIG. 10B, the tracking application 50 responds by loading a graphical representation 324 of that cart 100 on the GUI of the tracking application 50, thereby indicating that the cart 100 is ready to have one or more newly received cart inserts 110 assigned to the cart 100. The onscreen graphical representation 324 of the cart 100 displays a front view of the cart having the same number and configuration of drawers as that of the real cart 100 that is being represented thereby.

The nurse next scans the cart insert barcode on the retrieved cart insert 110. A validation is performed by the tracking application 50, by comparing data stored in the database 52 for that cart insert to data associated with that medication cart 100, to confirm that the cart insert 110 should be loaded into that specific cart 100. For example, the tracking application 50 may retrieve from the database 52 the internal location data assigned to that cart insert 110 by the pharmacy, and an internal location data assigned to the cart barcode of that medication cart 100. The tracking application 50 may then compare the two locations to ensure that they each list the same internal location. In other words, the tracking application is ensuring that the internal location data assigned to the cart insert 110 is the same as the internal location data assigned to the medication cart 100.

Once validated, resident names 326 associated with the cart insert 110 are displayed on the facility computing device 20, as depicted in FIG. 10C, and the nurse decides which drawer position into which to load the cart insert 110, and if necessary, opens the drawer 106 and removes a previously inserted cart insert 110 from the drawer 106. The removed cart insert can be recycled or sent back to the pharmacy to be reused in a subsequent order. The nurse next uses the input device 54, such as for example the handheld scanner, of the facility computing device 20 assigned to the cart 100, and scans a drawer position barcode affixed to a bottom of the selected drawer, at a position within the drawer into which the cart insert 110 is to be loaded. In one embodiment, in which a given drawer 106 can only accommodate one cart insert 110, there will only be a single barcode affixed to the bottom of that drawer 106. However, in alternate embodiments, a drawer 106 may be able to accommodate two or more drawer inserts located side-by-side within the drawer. In such an embodiment, a first drawer position barcode label will be affixed to the bottom of the drawer 106 on a left side of the drawer, and a second drawer position barcode label, having a different barcode than that of the first drawer position barcode label, will be affixed to the bottom of the same drawer 106 on a right side of the drawer 106 and to positioned to the right of the first drawer position barcode.

Referring to FIG. 10D, once both the cart insert barcode on the cart insert 110 and the drawer position barcode have been scanned, the tracking application 50 assigns or maps that cart insert 110 into that specific position in the drawer 106, overwriting the assignment of the previous removed cart insert to that drawer position, and displays the assignment 328 to the corresponding drawer. The nurse then places the new cart insert 110 into the drawer 106 at the assigned position. The nurse repeats this assignment and loading procedure for any additional cart inserts 110 received in the tote, or in additional totes, from the pharmacy until either the cart 100 is fully loaded with new cart inserts 110, or there are no more cart inserts 110 to load into the cart 100. For each such cart insert 110 loaded into a drawer 106 of the cart 100, the database 52 is updated for that cart insert to add in the specific drawer 106 and drawer position at which the cart insert 110 is located. In this manner, the database 52 now has a completely tracked accounting of which specific cart 100, and at which specific drawer position within the cart 100, a given cart insert 110 can be found. Accordingly, the database 52 also therefore has a full accounting as to where each slot location of each cart insert is within the cart, and thus the specific drawer and slot locations, in relation to the lights disposed about the edges of each drawer 106 of the cart 100, at which each medication of each resident is located.

Referring to FIGS. 10D and 10E, next the nurse proceeds to administer the medications to each resident at the internal location served by the cart 100. If necessary, the nurse again logs into the tracking application 50 on the facility computing device 20 that is assigned to a given cart 100 for an internal location of a facility, and clicks on a medication administering tab 323 (e.g., MedPass) to open or navigate to a medication administering page, tab, function, or workflow of the application 50 to initiate the medication administering workflow for the cart 100. The nurse uses the input device 54, such as a scanner, of the facility computing device 20 to scan the cart barcode of the specific medication cart 100 form which medications will be administered. Scanning the cart's 100 barcode triggers the tracking application 50 to retrieve from the database 52 a list of the residents having medications stored within the scanned cart 100 that are to be administered to the respective residents, as well as the dosages and other pertinent information, such as the time schedules at which such medications are to be administered. The nurse selects, via a computer mouse or otherwise on the GUI of the tracking application 50, a resident from the list provided to which medications are to be administered.

In response to selecting a resident to which medications are to be provided, a list of all such medications that are being taken by the resident is loaded onto the GUI of the tracking application 50. The listed medications are color coded based on the timing or schedule at which each is to be administered. In one embodiment, a listed medication that is color coded red indicates that the resident is past due to receive the medication. A listed medication that is color coded yellow indicates that the resident is due to receive the medication now. A listed medication that is color coded blue indicates that the resident is not yet due to receive the medication or that medication is due to be administered in the future. In alternate embodiments, alternate color codings may be utilized for a similar purpose without departing form the scope of the present disclosure.

The nurse selects in the GUI of the tracking application 52, via a computer mouse or other input device, one of the medications listed onscreen that is to be administered to the resident. The selection of the medication in the tracking application 50 triggers the application 50 to recall, from the database 52 of the medication tracking service 12 any/all of the assigned position data for that medication that was recorded upon the medication being assigned to cart insert 110 at the pharmacy, and upon the loading of the cart insert 110 containing the medication into a drawer position of the cart 100. Accordingly, the tracking application can recall any one or more of: the location of the slot within the cart insert 110 in which the medication is stored; the specific drawer 106 of the cart 100 in which the cart insert 110 has been loaded; the specific drawer position within the drawer 106 to which the cart insert 110 has been loaded. From this information, the tracking application 50 can also easily determine, where the medication is located within the cart, in relation to the lights 114 disposed at the front edges and side edges of the specific drawer in which the medication is located.

Accordingly, upon selecting the medication in the tracking application 50 and the recalling of the position data for the selected medication within the cart 100, as discussed above, the tracking application 50 further triggers, based on the recalled position data, the facility computing device 20 to send a lighting control signal to the light control module 122. The lighting control signal instructs the light control module 122 to illuminate only those lights 114 along the front edge and, in certain embodiments, the side edges of the drawer that are located adjacent the specific row and column of slots 112 in the cart insert that are aligned with the slot 112 of the cart insert 110 in which the resident's medication is stored. The illuminated lights 114 on the front edge of the drawers are visible whether the drawer is open or closed, and accordingly provides easy visual indication to the nurse as to which drawer to open to access the resident's selected medication. Once the nurse opens that specific drawer 106, the nurse is also then able to clearly see the illuminated lights 114 on both the front edges and the side edges of the drawer 106. The only lights 114 that are illuminated on the front and side edges of the drawer 106 are those lights 114 that are adjacent to the specific row and specific column of slots 112 in the cart insert 110 that are aligned with, and correspond to, the specific slot 112 at which the resident's medication are stored. In this manner, the single slot 112 found at the intersection of the lighted row of slots and the lighted column of slots is where the selected medication of the resident can be found, thus providing the nurse with an easy visual indication of the location of the medication to be administered to the selected patient

In some embodiments, the selection of the medication in the tracking application 50 also triggers the tracking application 50 to display in the onscreen GUI a graphic representation of a front view of the cart 100, and a separate top view graphic representation of the cart insert in which the medication can be found. The graphic representation of the front view of the cart has the same number and configuration of the drawers as is present in the real medication cart 100, and the graphic representation of the cart insert has the same number of and layout of available slots arranged into the same number of rows and columns as is found in the real cart insert 110. Tracking application 50 simultaneously highlights, in each of the respective onscreen representation of the cart and the cart insert, the specific drawer of the cart in which the selected medication can be found, as well as the specific slot of the cart insert in which the medication is stored. The highlighting can be done in any number of ways, including by, for example, changing the onscreen colors of the representations of each of the respective drawer and slot, as compared to the color of the remaining depicted drawers and slots, or by drawing a large thick border around the perimeter of each respective highlighted drawer and slot, or having the color of each such highlighted drawer and slot blink on/off or blink with changing colors, without departing form the scope of the present disclosure.

The method continues by the nurse retrieving the resident's medication from the slot found at the intersection of the row and column of slots illuminated by the lights 114, scanning the medication barcode on the medication packaging, and administering the medication to the resident, or alternatively placing the medication into a cup for administering together with additional medications that also need to be administered to the resident. The nurse then returns any unused or remaining portion of the medication to the slot from which it was removed, for future use. Scanning the medication barcode causes the corresponding onscreen color code for that medication in the tracking application 50 to turn green, thereby indicating that the medication was just administered. If there are multiple medications for the resident located within the slot that are either due or past due to be administered, the lights 114 will remain on and the nurse will also scan those medications and administer them to the resident as well, thus causing the corresponding color code for those additional medications listed on the GUI of the tracking application 50 to also turn green. Once all due, or past due, medications in a given slot 112 have been scanned and administered to a resident, the tracking application sends a signal via the facility computing device to the light control module to turn off the lights 114 and the drawer can be closed.

For each such remaining medication to be administered to the resident from the list of due or past due medications, the nurse repeats each of the steps of selecting such medications in the tracking application 50, opening the drawer 106 illuminated by the lights 114, identifying the slot 112 at which the medications are found based on the illuminated lights 114 aligned with the row and column of slots at which the slot 112 is found, scanning the medication, administering the medication, returning any unused medications back to the slot from which they were removed, and closing the drawer after the lights have turned off. The nurse also repeats this process for each remaining resident having medication stored in the cart, until all such medications have been administered to each resident of the internal location of the facility to be serviced by the cart.

While various of the aforementioned embodiments disclose a system and method that is based on barcodes and barcode scanning to track the packing, shipping, and receiving of medications, the loading of medications into medication carts, and the administering of medications to residents, in alternate embodiments the system and method can alternately be based on QR code and scanning technology, with barcodes and barcode scanners replaced with QR codes and QR code cameras or scanners without departing from the scope of the present disclosure. In still further embodiments, the system and method can also alternatively be based on RFID technology, in which the barcodes and/or barcode labels applied to each of the various medications, cart inserts, shipping totes, carts, and drawers are instead replaced with RFID chips or RFID labels, and the barcode scanners are replaced with RFID scanners, without departing from the scope of the present disclosure. In certain aspects, artificial intelligence (AI) applications may also be applied to ensure the verification and retrieval of the appropriate medication.

FIG. 12 is a block diagram illustrating an example computer system 1200 with which the at least one medication tracking service 12, the pharmacy computing device 16, the medication shipping computing device 18, and the at least one facility computing device 20 of FIG. 2 can be implemented. In certain aspects, the computer system 1200 may be implemented using hardware or a combination of software and hardware, either in a dedicated server, or integrated into another entity, or distributed across multiple entities.

Computer system 1200 (e.g., the at least one medication tracking service 12, the pharmacy computing device 16, the medication shipping computing device 18, and the at least one facility computing device 20) includes a bus 1208 or other communication mechanism for communicating information, and a processor 1202 (e.g., the processor 32, 36, 40, 44) coupled with bus 1208 for processing information. According to one aspect, the computer system 1200 can be a cloud computing server of an IaaS that is able to support PaaS and SaaS services.

Computer system 1200 can include, in addition to hardware, code that creates an execution environment for the computer program in question, e.g., code that constitutes processor firmware, a protocol stack, a database management system, an operating system, or a combination of one or more of them stored in an included memory 1204 (e.g., the memory 34, 38, 42, 46), such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device, coupled to bus 1208 for storing information and instructions to be executed by processor 1202. The processor 1202 and the memory 1204 can be supplemented by, or incorporated in, special purpose logic circuitry.

The instructions may be stored in the memory 1204 and implemented in one or more computer program products, e.g., one or more modules of computer program instructions encoded on a computer readable medium for execution by, or to control the operation of, the computer system 1200.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network, such as in a cloud-computing environment. The processes and logic flows described in this specification can be performed by one or more programmable processors executing one or more computer programs to perform functions by operating on input data and generating output.

Computer system 1200 further includes a data storage device 1206 such as a magnetic disk or optical disk, coupled to bus 1208 for storing information and instructions. Computer system 1200 may be coupled via input/output module 1210 to various devices. The input/output module 1210 can be any input/output module. Example input/output modules 1210 include data ports such as USB ports. In addition, input/output module 1210 may be provided in communication with processor 1202, so as to enable near area communication of computer system 1200 with other devices. The input/output module 1210 may provide, for example, for wired communication in some implementations, or for wireless communication in other implementations, and multiple interfaces may also be used. The input/output module 1210 is configured to connect to a communications module 1212. Example communications modules 1212 (e.g., the communications module 24, 26, 28, 30) include networking interface cards, such as Ethernet cards and modems.

In certain aspects, the input/output module 1210 is configured to connect to a plurality of devices, such as an input device 1214 and/or an output device 1216. Example input devices 1214 include a keyboard and a pointing device, e.g., a mouse or a trackball, by which a user can provide input to the computer system 1200. Other kinds of input devices 1214 can be used to provide for interaction with a user as well, such as a tactile input device, visual input device, audio input device, or brain-computer interface device.

According to one aspect of the present disclosure the at least one medication tracking service 12, the pharmacy computing device 16, the medication shipping computing device 18, and the at least one facility computing device 20 can be implemented using a computer system 1200 in response to processor 1202 executing one or more sequences of one or more instructions contained in memory 1204. Such instructions may be read into memory 1204 from another machine-readable medium, such as data storage device 1206. Execution of the sequences of instructions contained in main memory 1204 causes processor 1202 to perform the process steps described herein. One or more processors in a multi-processing arrangement may also be employed to execute the sequences of instructions contained in memory 1204. Processor 1202 may process the executable instructions and/or data structures by remotely accessing the computer program product, for example by downloading the executable instructions and/or data structures from a remote server through communications module 1212 (e.g., as in a cloud-computing environment). In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. For example, some aspects of the subject matter described in this specification may be performed on a cloud-computing environment. Accordingly, in certain aspects a user of systems and methods as disclosed herein may perform at least some of the steps by accessing a cloud server through a network connection. Further, data files, circuit diagrams, performance specifications and the like resulting from the disclosure may be stored in a database server in the cloud-computing environment, or may be downloaded to a private storage device from the cloud-computing environment.

The term “machine-readable storage medium” or “computer-readable medium” as used herein refers to any medium or media that participates in providing instructions or data to processor 1202 for execution. The term “storage medium” as used herein refers to any non-transitory media that store data and/or instructions that cause a machine to operate in a specific fashion. Such a medium may take many forms, including, but not limited to, non-volatile media, volatile media, and transmission media.

As used in this specification of this application, the terms “computer-readable storage medium” and “computer-readable media” are entirely restricted to tangible, physical objects that store information in a form that is readable by a computer. These terms exclude any wireless signals, wired download signals, and any other ephemeral signals. Storage media is distinct from but may be used in conjunction with transmission media. Transmission media participates in transferring information between storage media. For example, transmission media includes coaxial cables, copper wire and fiber optics, including the wires that comprise bus 1208. Transmission media can also take the form of acoustic or light waves, such as those generated during radio-wave and infra-red data communications. Furthermore, as used in this specification of this application, the terms “computer”, “server”, “processor”, and “memory” all refer to electronic or other technological devices. These terms exclude people or groups of people. For the purposes of the specification, the terms display or displaying means displaying on an electronic device.

In one aspect, a method may be an operation, an instruction, or a function and vice versa. In one aspect, a clause or a claim may be amended to include some or all of the words (e.g., instructions, operations, functions, or components) recited in either one or more clauses, one or more words, one or more sentences, one or more phrases, one or more paragraphs, and/or one or more claims.

To illustrate the interchangeability of hardware and software, items such as the various illustrative blocks, modules, components, methods, operations, instructions, and algorithms have been described generally in terms of their functionality. Whether such functionality is implemented as hardware, software or a combination of hardware and software depends upon the particular application and design constraints imposed on the overall system. Skilled artisans may implement the described functionality in varying ways for each particular application.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for”.

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

What is claimed is:

1. A method of tracking medications to be administered, comprising:
assigning, in a pharmacy computing device, a medication of a resident to a desired slot in a cart insert having a plurality of slots defined therein arranged into a plurality of rows and columns;
recording, in a database of a medication tracking service in communication with the pharmacy computing device, medication tracking data including a location of the assigned slot to which the medication was assigned in the cart insert, and an identification of the resident to whom the assigned medication belongs;
packing the first medication into the assigned slot of the cart insert;
allocating, in a facility computing device, the cart insert to a desired drawer position within a desired drawer of a medication cart;
recording, in the database in communication with the facility computing device, additional medication tracking data, including identification of each of the specific drawer of the cart and the drawer position within the cart to which the cart insert is allocated;
loading the cart insert into the drawer of the cart at the allocated drawer position; and
signaling the location at which the selected medication to be administered to the resident is located within the medical cart by:
transmitting the medication tracking data from the database to the facility computing device;
determining, by the facility computing device based on the medication tracking data, each of the drawer and drawer position in the cart in which the cart insert containing the mediation is loaded, the slot position within the cart insert into which the medication is packed, and a relative location of the slot in relation to a plurality of lights disposed along each of a front and side edge of the drawer in which the slot is located;
illuminating, by the facility computing device, a group of lights from each of the plurality of lights disposed along the font edge of the drawer that are visible when the drawer is in a closed position and a plurality of lights disposed along the side edge of the drawer, which illuminated group of lights are positioned at locations on the respective side and front edges of the drawer adjacent the respective row and column at which the slot is located in the cart insert within the drawer of the cart, at the intersection of which row and column the slot containing the medication is located.

2. The method of claim 1, wherein said assigning step comprises:
scanning, with a barcode scanner in communication with the pharmacy computing device, a medication barcode corresponding to the identification of the medication to be assigned to the desired slot and the resident to which the medication is to be administered; and
scanning, with the barcode scanner in communication with the pharmacy computing device, a slot position barcode corresponding to the identification of the location in the cart insert at which the slot is found; and
mapping, by the pharmacy computing device, the medication to the slot location.

3. The method of claim 2, further comprising:
inputting into the pharmacy computing device an identification of the specific cart insert into which the medication is to be assigned and packed, wherein the medication tracking data to be recorded in the database for each medication further includes the identification of the cart insert to which the medication was assigned.

4. The method of claim 3, wherein said inputting step comprises scanning, with a barcode scanner in communication with the pharmacy computing device, a cart insert barcode corresponding to the identification of the cart insert.

5. The method of claim 1, further comprising:
entering in the pharmacy computing device an internal location of a facility to which the cart insert packed with medication is to be shipped;
recording in the database, as additional medication tracking data for each medication assigned to a slot in the cart insert, the internal location of the facility to which the cart insert is to be shipped; and
shipping the cart insert to the internal location of the facility.

6. The method of claim 5, wherein said entering step comprises scanning, with a barcode scanner in communication with the pharmacy computing device, a location barcode corresponding to the first internal location of the facility.

7. The method of claim 6, wherein the location barcode corresponding to the first internal location of the facility is one of a plurality of location barcodes, printed in a master manual, that identify a plurality of available locations to which the cart insert may be shipped.

8. The method of claim 1, wherein the desired slot in the cart insert to which the medication is assigned is one of either an empty slot or a slot occupied by other medications for the resident, such that only medications of a single resident are located in any single slot of the cart insert.

9. The method of claim 1, wherein said assigning step further comprises:
generating on a screen of the facility computing device, a graphic representation of a top view of the cart insert having the same number and arrangement of slots as the real-world cart insert; and
displaying, by the pharmacy computing device, the name of the resident and medication information for the medication that was assigned to the slot, in the corresponding slot position of the onscreen graphic representation of the cart insert.

10. The method of claim 8, further comprising:
repeating each of the assigning, recording, and packing steps for any additional medications of either the resident and/or additional residents residing at a first internal location of the facility.

11. The method of claim 4, wherein said step of allocating the cart insert to a desired drawer position within a desired drawer of the medication cart comprises:
scanning, via a barcode scanner in communication with the facility computing device, a cart barcode affixed to a medication cart to input an identification of the specific medication cart to which the cart insert will be allocated;
scanning, via the barcode scanner, the cart insert barcode to input in the identification of the cart insert to be loaded into a drawer of the medication cart; and
scanning, via the barcode scanner, a drawer position barcode to input in the facility computing device the identification of both the drawer and position within in the drawer into which the cart insert is to be loaded, thereby allocating the cart insert to the scanned cart, drawer, and drawer position,
wherein the additional medication tracking data recorded in the database for each medication further includes the identification of the specific medication cart to which the cart insert will be allocated.

12. The method of claim 1, wherein said signaling step further comprises:
prior to said determining step, selecting in the facility computing device each of an identification of the resident to receive the medication stored within the cart, and the specific medication to be administered to the resident,
wherein the determination from said determining step is additionally based at least in part on the identification of the resident and the specific medication to be administered.

13. The method of claim 12, wherein said signaling step further comprises:
displaying on a screen of the facility computing device, graphic representation of a front view of the cart, and a separate graphic representation of a top view of the cart insert in which the medication can be found;
highlighting on each of the graphic representation of the front view of the cart and the graphic representation of the top view of the cart insert, the respective specific drawer corresponding to the real-world cart and the slot corresponding to the real-world slot in which the selected medication to be administered is located within the cart.

14. The method of claim 1, further comprising:
administering the selected medication to the resident from the identified drawer and identified slot.

15. A smart medication cart for use in administering medication to residents of a facility, comprising:
a cabinet defining a countertop;
a plurality of wheels affixed to a bottom of said cabinet;
a plurality of slidable drawers disposed within said cabinet and configured to be slidably moved between an opened position and a closed position, and to hold one or more selectively removable cart inserts having a plurality of rows and columns of medication storage slots defined therein; and
a plurality of lights disposed along a top surface of each of a front edge and a side edge of each of the plurality of drawers, and configured to be placed in communication with a facility computing device capable of tracking a drawer and slot location in a cart insert held in that drawer at which resident medications can be found, the lights being configured such that, upon selection in the facility computing device of a medication to be administered to a resident, the facility computing device selectively illuminates only the lights on the drawer disposed on the front and side edges that are aligned with the specific medication slot row and column at the intersection of which is located the slot containing the selected medications.

16. The smart medication cart of claim 15, further comprising:
a facility computing device in communication with the plurality of lights,
wherein the facility computing device is configured to allocate the one or more selectively removable cart inserts to a desired drawer position of the plurality of slidable drawers,
wherein the facility computing device is configured to receive, from a database in communication with the facility computing device, additional medication tracking data, including identification of each of the specific drawer of the cart and the drawer position within the cart to which the cart insert is allocated,
wherein the facility computing device is configured to signal a location at which a selected medication to be administered to a resident is located within the cabinet,
wherein the facility computing device is configured to receive the medication tracking data from the database,
wherein the facility computing device is configured to determine, based on the medication tracking data, each of the drawer and drawer position in the cart in which the cart insert containing the mediation is loaded, the slot position within the cart insert into which the medication is packed, and a relative location of the slot in relation to a plurality of lights disposed along each of a front and side edge of the drawer in which the slot is located, and
wherein the facility computing device is configured to illuminate a group of lights from each of the plurality of lights disposed along the font edge of the drawer that are visible when the drawer is in a closed position and a plurality of lights disposed along the side edge of the drawer, which illuminated group of lights are positioned at locations on the respective side and front edges of the drawer adjacent the respective row and column at which the slot is located in the cart insert within the drawer of the cart, at the intersection of which row and column the slot containing the medication is located.

17. The smart medication cart of claim 16, further comprising a barcode scanner in communication with the facility computing device,
wherein the facility computing device is configured to receive, from the barcode scanner, cart barcode information via the barcode scanner scanning a cart barcode affixed to a medication cart to input an identification of the specific medication cart to which the cart insert will be allocated,
wherein the facility computing device is configured to receive, from the barcode scanner, cart insert barcode information via the barcode scanner scanning a cart insert barcode to input in the identification of the cart insert to be loaded into a drawer of the medication cart, and
wherein the facility computing device is configured to receive, from the barcode scanner, drawer position barcode information via the barcode scanner scanning a drawer position barcode to input in the facility computing device the identification of both the drawer and position within in the drawer into which the cart insert is to be loaded, thereby allocating the cart insert to the scanned cart, drawer, and drawer position.

18. The smart medication cart of claim 17, wherein the facility computing device is configured to select each of an identification of the resident to receive the medication stored within the cart, and the specific medication to be administered to the resident.

19. The smart medication cart of claim 18, wherein the facility computing device is configured to display graphic representation of a front view of the cart, and a separate graphic representation of a top view of the cart insert in which the medication can be found, and
wherein the facility computing device is configured to highlight on each of the graphic representation of the front view of the cart and the graphic representation of the top view of the cart insert, the respective specific drawer corresponding to the real-world cart and the slot corresponding to the real-world slot in which the selected medication to be administered is located within the cart.

20. A computer-implemented method of tracking medications to be administered, comprising:
assigning, in a pharmacy computing device, a medication of a resident to a desired slot in a cart insert having a plurality of slots defined therein arranged into a plurality of rows and columns;
recording, in a database of a medication tracking service in communication with the pharmacy computing device, medication tracking data including a location of the assigned slot to which the medication was assigned in the cart insert, and an identification of the resident to whom the assigned medication belongs;
allocating, in a facility computing device, the cart insert to a desired drawer position within a desired drawer of a medication cart;
recording, in the database in communication with the facility computing device, additional medication tracking data, including identification of each of the specific drawer of the cart and the drawer position within the cart to which the cart insert is allocated; and
signaling the location at which the selected medication to be administered to the resident is located within the medical cart by:
transmitting the medication tracking data from the database to the facility computing device;
determining, by the facility computing device based on the medication tracking data, each of the drawer and drawer position in the cart in which the cart insert containing the mediation is loaded, the slot position within the cart insert into which the medication is packed, and a relative location of the slot in relation to a plurality of lights disposed along each of a front and side edge of the drawer in which the slot is located;
illuminating, by the facility computing device, a group of lights from each of the plurality of lights disposed along the font edge of the drawer that are visible when the drawer is in a closed position and a plurality of lights disposed along the side edge of the drawer, which illuminated group of lights are positioned at locations on the respective side and front edges of the drawer adjacent the respective row and column at which the slot is located in the cart insert within the drawer of the cart, at the intersection of which row and column the slot containing the medication is located.
